(19) United States
(12) Patent Application Publication
(10) Pub. No.: US 2025/0263451 A1
(43) Pub. Date: Aug. 21, 2025

(54) FUSION PROTEIN TARGETING MITOCHONDRIA, METHOD OF MAKING AND USE THEREOF

(71) Applicant: UNIVERSITY OF ROCHESTER, Rochester, NY (US)

(72) Inventors: Andrew Wojtovich, Rochester, NY (US); Brandon Berry, Auburn, NY (US); Shahaf Peleg, Rostock (DE)

(21) Appl. No.: 18/249,497

(22) PCT Filed: Nov. 17, 2021
(86) PCT No.: PCT/US2021/072461

§ 371 Date: Apr. 18, 2023

Related U.S. Application Data
(60) Provisional application No. 63/115,832, filed on Nov. 19, 2020.

(51) Int. Cl. C07K 14/37; A61K 38/00; A61K 48/00; A61N 5/06; C12N 9/02

(52) U.S. Cl. CPC C07K 14/37; A61K 48/0058; A61N 5/06; A61N 5/0622; C12N 9/001; A61K 38/00; A61N 2005/0662; C07K 2319/03; C07K 2319/07; C12Y 103/05001

(57) ABSTRACT

A fusion protein comprises (1) a first moiety that targets and orients the fusion protein to mitochondria inner membrane and (2) a second moiety that provides light-activated proton pump function when integrated into the mitochondria inner membrane. The fusion protein can be used for modulating hypoxia signaling in a subject, treating neurodegenerative diseases, protecting against stress, ameliorating symptoms of metabolic disorders and treating cancer.

This application is a national stage of International Application No. PCT/US21/72461, filed on Nov. 17, 2021, which claims priority of U.S. Provisional Application No. 63/115,832, filed on Nov. 19, 2020.

This invention was made with government support under NS092558, NS115906 and CA242843 awarded by the National Institutes of Health. The government has certain rights in the invention.

### FIELD

This application relates to the field of optogenetics and mitochondrial deactivation for therapeutic purposes, such as controlling cell functions, revitalizing cells and the amelioration of age-associated damage through optical control of mitochondrial metabolism.

### BACKGROUND

Mitochondria are semi-autonomous double-membrane-bound organelles found in most eukaryotic organisms. Mitochondria generate the bulk amount of cellular energy. Mitochondria generate an electrochemical proton gradient known as the protonmotive force (PMF). The PMF is like a battery, in that potential energy is stored for eventual release to do work. The PMF is created by the electron transport chain (ETC) in the mitochondrial inner membrane (IM) when electrons from metabolic substrates from food are passed along the chain and protons are pumped from the mitochondrial matrix to the intermembrane space (IMS) as oxygen is consumed.

Mitochondrial dysfunction is implicated in a wide range of disease. Optogenetics uses light-sensitive proteins to control biological functions. If targeted to mitochondria, optogenetic tools may allow rapid and precise manipulation of the PMF by controlled exposure to light. The ability to control mitochondrial function through use of light presents an intervention point to treat disease and other negative phenomenon.

### SUMMARY

One aspect of the present application relates to a fusion protein (mtOFF). The mtOFF fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix.

In some embodiments, the first moiety of the mtOFF fusion protein comprises an amino acid sequence selected from the group consisting of the mitochondria targeting sequence and transmembrane domains of one of human mitochondrial inner membrane protein (SDHC), rat SDHC, and mouse SDHC. In some embodiments, the first moiety comprises SEQ ID NO:6.

In some embodiments, the second moiety of the mtOFF fusion protein comprises an amino acid sequence selected from the group consisting of the protein sequence of Mac and variants, Arch and variants, bacteriorhodopsin (bR) and delta rhodopsin (dR). In some embodiments, the second moiety comprises SEQ ID NO:10.

In some embodiments, the first moiety is linked to the second moiety through a peptide linker. In some embodiments the peptide linker comprises the sequence of pro-ala-gly.

In some embodiments, the mtOFF fusion protein further comprises a third moiety that functions as a detection marker.

In some embodiments, the mtOFF fusion protein comprises an amino acid sequence that is at least 80% homologous to SEQ ID NO:6 and wherein the second moiety comprises an amino acid sequence that is at least 80% homologous to SEQ ID NO:10. In some embodiments, the mtOFF fusion protein comprises the amino acid sequence of SEQ ID NO:11.

Another aspect of the present application relates to a polynucleotide encoding the mtOFF fusion protein of the present application. In some embodiments, the polynucleotide comprises the nucleotide sequence of SEQ ID NO:12.

Another aspect of the present application relates to an expression cassette comprising a polynucleotide encoding the mtOFF fusion protein and a regulatory sequence operably linked to the polynucleotide.

Another aspect of the present application relates to an expression vector comprising a polynucleotide encoding the mtOFF fusion protein.

Another aspect of the present application relates to a mitochondrion containing the mtOFF fusion protein.

Another aspect of the present application relates to a cell containing mitochondria that contain the mtOFF fusion protein.

Another aspect of the present application relates to a pharmaceutical composition comprising an expression vector capable of expressing the mtOFF fusion protein and a pharmaceutically acceptable carrier

Another aspect of the present application relates to a method of treating or ameliorating symptoms of neurodegenerative diseases in a subject, comprising the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix; exposing the target cells to light to activate the proton pump to decrease the protonmotive force (PMF) across the mitochondrial inner membrane, wherein decreased PMF in the mitochondria of the target cells prevents development of symptoms, or ameliorates existing symptoms, of neurodegenerative diseases. In some embodiments, the target cells are neuronal cells. In some embodiments, the fusion protein is expressed in the target cells by infecting the target cells with a viral vector capable of expressing the fusion protein in the target cells.

Another aspect of the present application relates to a method of enhancing cell resistance to hypoxia in a subject, comprising the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix; exposing the target cells to light to activate the proton pump to decrease the protonmotive force (PMF) across the mitochondrial inner membrane, wherein decreased PMF in the mitochondria of the target cells enhances the target cells' resistance to hypoxia. In some embodiments, the target cells are neuronal cells. In some embodiments, the fusion protein is expressed in the target cells by infecting the target cells with a viral vector capable of expressing the fusion protein in the target cells.

Another aspect of the present application relates to a method of enhancing cell resistance to stress in a subject, comprising the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix, wherein the first moiety comprises a targeting/orienting sequence from SDHC; exposing the target cells to light to activate the proton pump to decrease the protonmotive force (PMF) across the mitochondrial inner membrane, wherein decreased PMF in the mitochondria of the target cells enhances the target cells' resistance to stress. In some embodiments, PMF in the mitochondria of the target cells is decreased to an extent that results in mitochondria autophagy. In some embodiments, the light-activated proton pump in the second moiety is a Mac proton pump.

Another aspect of the present application relates to a method of treating or ameliorating symptoms of metabolic disorders caused by mitochondrial dysfunction in a subject, comprising the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix, wherein the first moiety comprises a targeting/orienting sequence from SDHC; exposing the target cells to light to activate the proton pump to decrease the protonmotive force (PMF) across the mitochondrial inner membrane, wherein decreased PMF in the mitochondria of the target cells prevents development symptoms, or ameliorating existing symptoms, of the metabolic disorder. In some embodiments, PMF in the mitochondria of the target cells is decreased to an extent that results in mitochondria autophagy. In some embodiments, the light-activated proton pump in the second moiety is a Mac proton pump.

Another aspect of the present application relates to a method of treating cancer in a subject suffering from cancer, comprising the steps of: expressing a fusion protein in target cancer cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix; exposing target cancer cells to light to activate the proton pump to decrease the protonmotive force (PMF) across the mitochondrial inner membrane, wherein decreased PMF in the mitochondria of the target cancer cells inhibits cancer cell growth in the subject. In some embodiments, PMF in the mitochondria of the target cancer cells is decreased to an extent that results in mitochondria autophagy.

### BRIEF DESCRIPTION OF THE DRAWINGS

FIG. 1 is a composite of drawings and pictures showing mitochondria-OFF (mtOFF) decreases mitochondrial protonmotive force (PMF). Panel A) Schematic of mtOFF targeted to the mitochondrial inner membrane (IM) to dissipate the protonmotive force (PMF). Electron transport chain (ETC) complexes together consume 02 and generate the PMF by proton (H⁺) pumping from the matrix to the intermembrane space (IMS). Mitochondrial ATP synthase uses the PMF to make ATP from ADP. The N terminal mitochondria targeting sequence and two transmembrane regions of the rat SDHC protein are shown in pink fused to the red proton pumping portion of mtOFF. The red fluorescent protein mKate is shown on the C terminus in the IMS in red. Light activation of mtOFF results in proton pumping from the IMS to the matrix. Panel B) Fluorescent images show muscle mitochondria of a living C ubiquitously expressing mtOFF. Red signal shows mKate fluorescence and green signal shows MITOTRACKER™ Green staining of mitochondria. The merged image shows the mitochondrial localization of the mtOFF::mKate construct overlapping with MITOTRACKER™ Green signal. Scale bars are 5 μm. Panel C) Quantification of TMRE fluorescence intensity in isolated mitochondria incubated with succinate to fuel membrane potential (ΔΨm) shows decreased AI'm upon mtOFF activation. Proton pumping activity of mtOFF requires light and the cofactor all trans-retinal (ATR). Because C. elegans do not produce ATR endogenously, exogenous supplementation is required for the light-activated proton pump to function. Data are normalized to dark conditions and full light doses are presented in FIG. 2, panel B. One-way ANOVA was performed with Tukey's test for multiple comparisons, *p=0.0247. Data are means±SEM, n=4 independent mitochondrial isolations. Panel D) Quantification of change in BCECF-AM ratio in isolated mitochondria fueled with succinate shows decreased mitochondrial matrix pH after mtOFF activation. One-way ANOVA was performed with Tukey's test for multiple comparisons, −ATR −light vs. +ATR +light *p=0.0002, +ATR −light vs. +ATR +light *p=0.0002, −ATR +light vs. +ATR +light *p=0.0036. Data are means±SEM, n=4-5 independent mitochondria isolations. Panel E) O₂ consumption rates of whole animals normalized to dark conditions were increased upon mtOFF activation. Raw O₂ consumption rates are shown in FIG. 2, panel D. Two-tailed unpaired t test was performed, *p=0.0195. Data are means±SEM, n=5, where one n is one O₂ consumption rate from ˜1500 animals in a Clark type O₂ electrode. Panel F) Relative ATP levels normalized to dark conditions from whole animals was decreased upon mtOFF activation. Two-tailed unpaired t test was performed, *p=0.0230. Data are means±SEM, n=5 independent assays from three plates each for each condition containing at least 100 animals.

FIG. 2 is a composite of drawings and pictures showing mtOFF decreases the PMF. Panel A) Schematic showing mtOFF decreasing both components of the protonmotive force (PMF), the ΔΨm and the ΔpH, upon light exposure. mtOFF pumps protons (H+) from the intermembrane space (IMS) across the inner membrane (IM) into the matrix. Panel B) TMRE fluorescence was measured in response to increasing light doses. Increasing fluence (light dose, Joules/cm²) results in progressively decreased PMF in isolated mitochondria supplied with succinate. Data from 0 and 0.15 J/cm² are presented in FIG. 1, panel C. Two-way ANOVA with Holm-Sidak test for multiple comparisons was performed, 0.08 J/cm² *p=0.0068, 0.15 J/cm² *p=0.018, n=4 independent mitochondrial isolations. Data are means±SEM. Panel C) Representative BCECF-AM fluorescence ratio trace. Baseline level of mitochondria supplied with succinate from animals with and without ATR is shown followed by light treatment (no BCECF-AM fluorescence measured), and signal immediately after illumination. Mitochondria with ATR have a decreased matrix pH, indicating proton entry through mtOFF during light exposure. Rapid reestablishment of baseline pH shows the reversibility of mtOFF when light is removed. FCCP was then added to establish minimum signal. Panel D) Raw O₂ consumption values under baseline, light treatment, maximal respiration, and minimum respiration states in whole animals. Maximum respiration was induced by FCCP treatment, and minimum respiration was induced with azide treatment. Data are presented for animals with and without ATR and are means±SEM, n=5, where one n is one O₂ consumption rate from ˜1500 animals in a Clark type O₂ electrode. Normalized baseline data are presented in FIG. 1, panel E) Oil Red O density was quantified (a.u. is arbitrary units) in whole animals stained immediately after 10 minute activation of mtOFF, as performed in FIG. 1E and FIG. 2, panel D. mtOFF had no effect on fat stores. One way ANOVA was performed, p>0.05. Data are means±SEM.

FIG. 3 is a composite of drawings and pictures showing mtOFF modulates energy sensing behavior through AMPK. Panel A) Schematic showing locomotion differences in C. elegans under both fed (left) and starved (right) conditions. Removal from food results in increased locomotion, mediated by AMPK signaling. This output is used in this study to validate functional AMPK signaling. Panel B) Locomotion was scored by counting body bends per minute. Animals were scored either on food or immediately after being transferred off of food. C. elegans AMPK is encoded by the aak-2 gene. The non-functional aak-2 (ok524) mutant strain was used for AMPK mutant animals. One-way ANOVA with Tukey's test for multiple comparisons was performed, wild-type on food vs wild-type off of food *p<0.0001, wild-type off of food vs. AMPK mutant off of food *p<0.0001. Data are means±standard deviation, n=30-60 animals each condition from at least two experimental days. Panel C) Immunoblot against phosphorylated (active) AMPK (pAMPK, top bands, 62 kDa) and actin (bottom bands, 43 kDa) from whole animal lysate on the same blot shows increased phosphorylation level under conditions of activated mtOFF. Panel D) Quantification of pAMPK/actin densitometry shows increased pAMPK in response to mtOFF activation. pAMPK/actin is used to measure activated AMPK as there is no validated total AMPK antibody in C. elegans. One-way ANOVA with Tukey's test for multiple comparisons was performed, −ATR −light vs. +ATR +light *p=0.0001, +ATR −light vs. +ATR +light *p=0.0006, −ATR +light vs. +ATR +light *p=0.0043. Data are means±SEM, n=4 independent blots from one plate of worms for each condition per replicate, with at least 1000 animals per plate. Panel E) Schematic showing effects of mtOFF activation on locomotion. mtOFF is expected to create an energy demand through PMF dissipation that will increase locomotion, mediated by AMPK signaling. Panel F) Body bends were scored, and illumination was throughout measurement where indicated. Animals were exposed to 50 μM compound C, an AMPK inhibitor, for 24 hours where indicated. One-way ANOVA with Tukey's test for multiple comparisons was performed, −ATR +light vs. +ATR +light *p<0.0001, +ATR +light vs. +ATR +light +compound C *p<0.0001, +ATR +light vs. mtOFF x AMPK mutant +ATR +light +compound C *p<0.0001. n.s. is not significant, p=0.3999. Data are means±standard deviation, n=30-60 animals each condition from at least two experimental days. Panel G) Full length blots for FIG. 3, panel C immunoblot. Each blot shows separate biological replicates.

FIG. 4 is a composite of drawings and pictures showing mtOFF triggers neuronal AMPK to control locomotion. Panel A) Locomotion was scored by counting body bends per minute. Animals were scored either on food or immediately after being transferred off of food. All animals are expressing mtOFF, the center pair of bars are AMPK mutant animals, and the last pair of bars are AMPK mutant animals with functional AMPK re-expressed in neurons alone. AMPK mutant animals have a blunted response to starvation, and neuronal AMPK is sufficient to restore the response. One-way ANOVA with Tukey's test for multiple comparisons was performed, mtOFF off of food vs mtOFF x AMPK mutant off of food *p<0.0001, mtOFF x AMPK mutant off of food vs neuronal AMPK rescue *p<0.0001. n.s. is not significant, p=0.9910. Data are means±standard deviation, n=30-60 animals each condition. Data were collected on at least two different experimental days for each condition. Panel B) Locomotion in AMPK mutant animals expressing mtOFF with functional AMPK expressed only in neurons. Illumination was throughout measurement where indicated. mtOFF activation increased locomotion with functional AMPK expressed in neurons. One-way ANOVA with Tukey's test for multiple comparisons, −ATR +light vs. +ATR +light, *p<0.0001. Data are means±standard deviation, n=30-60 animals each condition from at least two experimental days. Panel C) Schematic showing tissue-specific mtOFF expression in neurons alone or in intestine alone. Neurons are responsible locomotion response to food sensation in an AMPK dependent manner. mtOFF was expressed in neurons to test if PMF loss in neurons alone could mediate an increased locomotion response. Intestine is the organ that absorbs nutrients, and mtOFF was expressed here to rule out the role of intestinal control of locomotion in response to PMF loss. Panel D) Locomotion in animals expressing mtOFF only in neurons. Illumination was throughout body bends measurement where indicated. mtOFF activation in neurons increased locomotion compared to controls. Light alone also increased locomotion. One-way ANOVA with Tukey's test for multiple comparisons was performed, −ATR vs. −ATR +light *p=0.0251, +ATR −light vs. −ATR +light *p<0.0083, −ATR +light vs. +ATR +light *p<0.0001. Data are means±standard deviation, n=30-60 animals each condition from at least two experimental days. Panel E) Locomotion in animals expressing mtOFF only in intestine. Illumination was throughout body bends measurement where indicated. mtOFF activation in intestine did not increase locomotion compared to controls. Light alone also increased locomotion, similar to panel D. One-way ANOVA with Tukey's test for multiple comparisons was performed, −ATR −light vs. −ATR +light *p=0.0007, −ATR −light vs. +ATR +light *p=0.0019. n.s. is not significant, p=0.9910. Data are means±standard deviation, n=30-60 animals each condition from at least two experimental days.

FIG. 5 is a composite of drawings and pictures showing mtOFF protects against hypoxia through neuronal AMPK. Panel A) Schematic showing protocol to activate mtOFF before hypoxia exposure to test the prophylactic effects of PMF dissipation on hypoxia resistance. Control conditions with and without ATR were either treated with light or left in the dark before hypoxia exposure. Normoxia is denoted with an open bar, and hypoxia is denoted by a striped bar. Timeline not to scale. To assess protection against hypoxia, the percent survival (alive/(alive+dead)*100%) under-light conditions was subtracted from percent survival under +light conditions with and without ATR (top bar subtracted from bottom bar). The resulting protection percent (%) would be negative for damaging interventions after hypoxia exposure, and positive for interventions protective against hypoxia. Experiments are paired by concurrent hypoxia exposure. Panel B) mtOFF activation prior to hypoxia conferred protection. Two-tailed paired t test, *p=0.0082. Data are means±SEM, n=5, where one n is an average of three technical replicates of plates containing 15-50 animals. Panel C) AMPK mutant animals expressing mtOFF were not protected against hypoxia, suggesting the protection observed in panel B requires AMPK signaling. Two-tailed paired t test was performed, n.s. is not significant, p=0.176. Data are means±SEM, n=4-5, where one n is an average of three technical replicates of plates containing 15-50 animals. Panel D) AMPK mutant animals expressing mtOFF with functional AMPK expressed in neurons alone conferred protection against hypoxia, suggesting AMPK activity in neurons alone is sufficient for the hypoxia resistance triggered by decreased PMF. Two-tailed paired t test was performed, *p=0.0069. Data are means±SEM, n=3, where one n is an average of three technical replicates of plates containing 15-50 animals. Panel E) Intestinal mtOFF activation prior to hypoxia conferred protection. Two-tailed paired t test was performed, n.s. is not significant, p=0.645. Data are means±SEM, n=3, where one n is an average of three technical replicates of plates containing 15-50 animals.

FIG. 6 is a composite of drawings and pictures showing mitochondrial localization of mtOFF. Panel A) Schematic showing expected fluorescence localization in single mitochondria for mtOFF::mKate, intermembrane space (IMS)::GFP, and MITOTRACKER™ Green. Bottom row shows the expected fluorescence pattern of merged images. Panel B) Fluorescent images of muscle mitochondria in live C. elegans coexpressing IMS::GFP and mtOFF::mKate (left) or expressing mtOFF::mKate and stained with MITOTRACKER™ Green. Scale bars are 5 μm. Panel C) Representative profile fluorescence intensity plots for single mitochondria from the images in panel B. The white letter d shows the distance between inflection points of the red and green fluorescent signals. Panel D) The distance between inflection points was quantified (examples shown in panel d). mtOFF::mKate localized close to IMS::GFP signal, and distant from the matrix MITOTRACKER™ Green signal as expected, with the C terminal mKate predicted to be in the IMS. Two-tailed unpaired t test was performed, *p=0.0137. Data are means±SEM, n=14 mitochondria from distinct animals for each condition.

FIG. 7 shows mtOFF starved locomotion. mtOFF activation in animals off of food resulted in a small but significant decrease in locomotion. Data was collected on at least two different experimental days for each condition. One way ANOVA with Tukey's test for multiple comparisons was performed, −ATR −light vs. +ATR +light p<0.0001, +ATR −light vs. +ATR +light p=0.0056, −ATR +light vs. +ATR +light *p=0.0008, n=30-60 animals each condition. Data are means±standard deviation.

FIG. 8 is a composite of drawings showing raw survival data following hypoxia for FIG. 5. These raw data were used to calculate protection % in FIG. 5 to account for ATR and light exposure effects. See FIG. 5 legend and methods section for details on statistics. For each set of experiments, n=3, where one n is an average of three technical replicates of plates containing 15-50 animals. Data are means±SEM. Panel A) Survival % for animals expressing mtOFF after hypoxia exposure. Protection % presented in FIG. 5, panel B) Survival % for animals expressing mtOFF in the AMPK mutant background after hypoxia exposure. Protection % presented in FIG. 5, panel C) Survival % for animals expressing mtOFF in the AMPK mutant background with AMPK expression rescued in neurons after hypoxia exposure. Protection % presented in FIG. 5, panel D) Survival % for animals expressing mtOFF in intestine after hypoxia exposure. Protection % presented in FIG. 5, panel E.

FIG. 9 shows mtOFF activation results in oxidative stress resistance. Day 1 adults were exposed to 200 mM paraquat. Continuous mtOFF activation resulted in increased survival over time. Two-way ANOVA with Tukey's test for multiple comparisons was performed. For hour 3, −ATR −light vs. +ATR +light *p<0.0001, +ATR −light vs. +ATR +light *p=0.0297, −ATR −light vs. −ATR +light *p=0.0024. For hour 4, −ATR −light vs. +ATR +light *p<0.0001, +ATR −light vs. +ATR +light *p<0.0001, −ATR +light vs. +ATR +light *p<0.0001. Data are means±SEM, n=3 biological replicates with at least 30 animals for each condition.

While the present disclosure will now be described in detail, and it is done so in connection with the illustrative embodiments, it is not limited by the particular embodiments illustrated in the figures and the appended claims.

### DETAILED DESCRIPTION

Herein incorporated by reference is the sequence listing filed with the USPTO named as 1134-111 NATL ST25.txt which was created on Nov. 6, 2023, and the size of the TXT file is 24,797 in bytes.

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

### Definitions and Terminology

As used herein, the following terms shall have the following meanings:

The term “polynucleotide” refers to deoxyribonucleotides or ribonucleotides and polymers thereof in either single- or double-stranded form. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. The term “polynucleotide” or “polynucleotide sequence” can also be used interchangeably with gene, open reading frame (ORF), cDNA, and mRNA encoded by a gene.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term “variant” refers to protein or polypeptide that is different from the reference protein or polypeptide by one or more amino acids, e.g., one or more amino acid substitutions, but substantially maintains the biological function of the reference protein or polypeptide. The term “variant” further includes conservatively substituted variants. The term “conservatively substituted variant” refers to a peptide comprising an amino acid residue sequence that differs from a reference peptide by one or more conservative amino acid substitution and maintains some or all of the activity of the reference peptide as described herein. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another, such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. The phrase “conservatively substituted variant” also includes peptides wherein a residue is replaced with a chemically derivatized residue, provided that the resulting peptide maintains some or all of the activity of the reference peptide as described herein. In some embodiments, the functional variant of a peptide shares a sequence identity of 70%, 75%, 80%, 85%, 90%, 91%, 92%, 93%, 94%, 95%, 96%, 97%, 98% or 99% with the reference peptide. For example, a functional variant of a protein may share a sequence identity of 70%, 75%, 80%, 85%, 90%, 91%, 92%, 93%, 94%, 95%, 96%, 97%, 98% and 99% with the reference version of the protein; and a functional variant of a fusion protein may share a sequence identity of 70%, 75%, 80%, 85%, 90%, 91%, 92%, 93%, 94%, 95%, 96%, 97%, 98% and 99% with the reference fusion protein.

A variant of a polypeptide may be a fragment of the original polypeptide. The term “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 3, 5, 6, 8 or 10 amino acids long, at least 14 amino acids long, at least 20, 30, 40 or 50 amino acids long, at least 75 amino acids long, or at least 100, 150, 200, or more amino acids long.

The term “homologous amino acid sequence” used in this specification, unless otherwise stated herein, refers to an amino acid sequence derived from the substitution of one or more amino acids in the amino acid sequence of a polypeptide. Furthermore, the term “homologous polypeptide” used in this specification, unless otherwise stated herein, refers to a polypeptide homologue derived from the substitution of one or more amino acids in the amino acid sequence of a polypeptide.

The term “sequence identity,” as used herein, means that two peptide sequences are identical (i.e., on an amino acid-by-amino acid basis) over the window of comparison. The term “percentage of sequence identity” is calculated by comparing two optimally aligned sequences over the window of comparison, determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity. The reference sequence may be a subset of a larger sequence, for example, as a segment of the full-length sequences of the compositions claimed in the present invention.

The term “proton pump” means an integral membrane protein that is capable of moving protons across the membrane of a cell, mitochondrion, or other subcellular compartment. For example, bacteriorhodopsins are light-activated electrogenic proton pumps that are 7-transmembrane helix proteins (7-TM), utilize all-trans retinal as their chromophore in their native state, and bear structural similarity to the H. salinarum bacteriorhodopsin. Commonly characterized bacteriorhodopsins are the H. salinarum bacteriorhodopsin, the S. ruber xanthorhodopsin, and uncultured gamma-protobacterium BAC31A8. Other examples are microbial rhodopsins, such as the Halorubrum sodomense gene for archaerhodopsin-3 (herein abbreviated “Arch”) and Halorubrum strain TP009 gene for archaerhodopsin-TP009 (herein abbreviated “ArchT”), and eukaryotic proton pumps, such as leptosphaeria maculans (herein abbreviated “Mac”), P. triticirepentis and S. sclerotorium rhodopsins.

The term “expression cassette,” as used herein, refers to a DNA or RNA construct that contains one or more transcriptional regulatory elements operably linked to a nucleotide sequence coding the fusion protein of the present application. An expression cassette may additionally contain one or more elements positively affecting mRNA stability and/or an internal ribosome entry site (IRES) between adjacent protein coding regions to facilitate expression two or more proteins from a common mRNA.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

The term “regulatory elements” refers to DNA/RNA sequences necessary for the expression of an operably linked coding sequence in one or more host organisms. The term “regulatory elements” is intended to include promoters, enhancers and other expression control elements (e.g., polyadenylation signals). Regulatory elements include those which direct constitutive expression of a nucleotide sequence in many types of host cells or those which direct expression of the nucleotide sequence only in certain host cells (e.g., tissue-specific regulatory elements). Expression cassettes generally contain sequences for transcriptional termination, and may additionally contain one or more elements positively affecting mRNA stability.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

The term “expression vectors,” as used herein, refers to recombinant expression vectors comprising nucleic acid molecules which encode the fusion proteins disclosed herein. Particularly useful vectors are contemplated to be those vectors comprising the expression cassette of the present application or those vectors in which the coding portion of the DNA segment is positioned under the control of a regulatory element. The expression vectors of the present application is capable of expressing the fusion protein of the present application in a cell transfected or infected by the expression vector. Expression vectors include non-viral vectors and viral vectors.

The term “non-viral vector,” as used herein, refers to an autonomously replicating, extrachromosomal circular DNA molecules, distinct from the normal genome. For example, a plasmid is a non-viral vector.

The terms “viral vector” and “recombinant virus” are used interchangeably herein to refer to any of the obligate intracellular parasites having no protein-synthesizing or energy-generating mechanism. The viral genome may be RNA or DNA contained with a coated structure of protein of a lipid membrane. The viruses useful in the practice of the present invention include recombinantly modified enveloped or non-enveloped DNA and RNA viruses, preferably selected from baculoviridiae, parvoviridiae, picornoviridiae, herpesviridiae, poxviridae, or adenoviridiae. The viral genomes may be modified by recombinant DNA techniques to include expression of exogenous transgenes and may be engineered to be replication deficient, conditionally replicating or replication competent. Chimeric viral vectors which exploit advantageous elements of each of the parent vector properties may also be useful in the practice of the present application. Minimal vector systems in which the viral backbone contains only the sequences need for packaging of the viral vector and may optionally include a transgene expression cassette may also be produced according to the practice of the present application. Although it is generally favored to employ a virus from the species to be treated, in some instances it may be advantageous to use vectors derived from different species which possess favorable pathogenic features. A viral vector may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like.

The term “retrovirus” refers to double-stranded RNA enveloped viruses that are primarily characterized by the ability to “reverse transcribe” their genome from RNA to DNA. The virions are 100-120 nm in diameter and contain a dimeric genome of the same plus RNA strand complexed with the nucleocapsid protein. The genome is encapsulated in a proteic capsid that also contains the enzymatic proteins required for viral infection, namely reverse transcriptase, integrase and protease. Matrix proteins form the outer layer of the capsid core that surrounds the viral nuclear particle and interacts with the envelope, a lipid bilayer derived from the host cell membrane. Immobilized in this bilayer is a viral envelope glycoprotein that is responsible for recognizing specific receptors on the host cell and initiating the infectious process. Envelope proteins are formed by two subunits, a transmembrane (TM) that anchors the protein within the lipid membrane and a surface (SU) that binds to cell receptors.

Based on the genomic structure, retroviruses are classified into simple retroviruses such as MLV and murine leukemia virus; or complex retroviruses such as HIV and EIAV. Retroviruses encode four genes, gag (group-specific antigen), pro (protease), pol (polymerase) and env (envelope). The gag sequence encodes three major structural proteins: matrix protein, nucleocapsid protein, and capsid protein. The pro sequence encodes a protease responsible for cleaving Gag and Gag-Pol during particle assembly, budding and maturation. The pol sequence encodes the enzymes reverse transcriptase and integrase, the former catalyzing the reverse transcription of the viral genome from RNA to DNA during the infection process and the latter the role of incorporating proviral DNA into the host cell genome. Carry. The env sequence encodes both the SU and TM subunits of the envelope glycoprotein. In addition, the retroviral genome contains two LTRs (long terminal repeats) that contain the elements necessary to facilitate gene expression, reverse transcription and integration into the host cell chromosome; viral RNA into newly formed virions. A sequence designated as the packaging signal (Y) required for specific packaging; as well as a non-coding cis such as a polypurine tract (PPT) that functions as a site to initiate plus-strand DNA synthesis during reverse transcription. The acting sequence is presented. In addition to gag, pro, pol and env, complex retroviruses such as lentiviruses regulate viral gene expression, assembly of infectious particles and modulate vif, vpr, vpu, nef, which modulates viral replication in infected cells. It has accessory genes including tat and rev.

During the process of infection, retroviruses first attach to specific cell surface receptors. Upon entry into a susceptible host cell, the retroviral RNA genome is copied into DNA by the virally encoded reverse transcriptase carried within the parental virus. This DNA is transported to the host cell nucleus and then integrated into the host genome. At this stage it is typically called a provirus. Proviruses are stable in the host chromosome during cell division and are transcribed like other cellular proteins. Proviruses encode the proteins and packaging machinery required to make more virus and can leave the cell by a process known as “budding”.

The term “lentivirus” or “lentiviral vector” as used herein, refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “adeno-associated virus (AAV)” or “recombinant AAV (rAAV),” as used herein, refers to a group of replication-defective, nonenveloped viruses, that depend on the presence of a second virus, such as adenovirus or herpes virus or suitable helper functions, for replication in cells. AAV is not known to cause disease and induces a very mild immune response. AAV can infect both dividing and non-dividing cells and may incorporate its genome into that of the host cell. More than 30 naturally occurring serotypes of AAV are available. Many natural variants in the AAV capsid exist, allowing identification and use of AAV vectors with properties specifically suited for the cell targets of delivery. AAV vectors are relatively non-toxic, provide efficient gene transfer, and can be easily optimized for specific purposes. AAV viruses may be engineered using conventional molecular biology techniques to optimize the generation of recombinant AAV particles for cell specific delivery of the fusion proteins, for minimizing immunogenicity, enhancing stability, delivery to the nucleus, etc.

The term “Car-T” refers to T cells modified to express a chimeric antigen receptor (CAR). T cells that have been genetically modified to express a CAR are used in treatments for cancers where the CAR redirects the modified T cell to recognize a tumor antigen. In some instances, it is beneficial to effectively control and regulate CAR T cells such that they kill tumor cells while not affecting normal bystander cells. The nucleic acid encoding CAR can be introduced into cells such as T cells using the retroviral vector or lentiviral vector. In this way, large numbers of cancer-specific T cells can be generated for adoptive cell transplantation methods. When CAR binds to the target antigen, an activating signal is transmitted to the T cells in which it is expressed. Thus, CAR dictates T cell specificity and cytotoxicity for tumor cells expressing the target antigen.

The term “mtOFF,” “mtOFF construct,” or “mtOFF protein” as used herein, refers to a fusion protein that comprises a first moiety that targets the fusion protein to the mitochondrial inner membrane and a second moiety that comprises a light-activated proton pump, wherein the first moiety orients the second moiety in a direction that allows the proton pump to pump protons from the inner membrane space to the mitochondrial matrix (mtOFF direction).

The term “mtOFF polynucleotide,” as used herein, refers to a polynucleotide comprising a sequence that encodes a mtOFF protein.

The term “mtOFF expression cassette,” as used herein, refers to an expression cassette comprising a mtOFF polynuclotide.

The term “mtOFF expression vector,” as used herein, refers to an expression vector capable of expressing a mtOFF protein inside a cell.

The term “mtOFF mitochondria,” as used herein, refers to mitochondria comprising one or more functional mtOFF proteins on their inner membrane.

The term “mtOFF cell,” as used herein, refers to a cell comprising one or more mtOFF mitochondria.

The term “mitochondrial autophagy” (mitophagy) as used herein refers to selective sequestration of mitochondria by autophagosomes, which subsequently deliver them to lysosomes for destruction. This process is essential for myocardial homeostasis and adaptation to stress. Elimination of damaged mitochondria protects against cell death, as well as stimulates mitochondrial biogenesis.

The terms “treat,” “treating” or “treatment” as used herein, refers to a method of alleviating or abrogating a disorder and/or its attendant symptoms. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

A “therapeutically effective amount,” as used herein, refers to an amount effective, at dosages and for periods of time necessary, to achieve the desired therapeutic result. A therapeutically effective amount of an expression vector may vary depending on the condition to be treated, the severity and course of the condition, the mode of administration, whether the agent is administered for preventive or therapeutic purposes, the bioavailability of the particular agent(s), the ability of the fusion protein or vector to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the expression vector is outweighed by the therapeutically beneficial effects. A “prophylactically effective amount” refers to an amount effective, at dosages and for periods of time necessary, to achieve the desired prophylactic result.

As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

The term “tumor” as used herein refers to a neoplasm or a solid lesion formed by an abnormal growth of cells. A tumor can be benign, pre-malignant or malignant.

The term “cancer” is defined as a malignant neoplasm or malignant tumor and is a class of diseases in which a group of cells display uncontrolled growth, invasion that intrudes upon and destroys adjacent tissues, and sometimes metastasis, or spreading to other locations in the body via lymph or blood. These three malignant properties of cancers differentiate them from benign tumors, which do not invade or metastasize. Exemplary cancers include: carcinoma, melanoma, sarcoma, lymphoma, leukemia, germ cell tumor, and blastoma.

As used herein, the term “inflammatory disorder” includes diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cell proliferative disorders, or other agents.

As used herein, the term “subject” includes both human and animal subjects. Thus, veterinary therapeutic uses are provided in accordance with the presently disclosed subject matter.

Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of fish and birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like, and treatment for fish.

The term “mammal” refers to any animal classified as a mammal, including humans, non-human primates, domestic and farm animals, and zoo, sports, or pet animals, such as dogs, horses, cats, cows, etc. Preferably, the mammal is human.

mtOFF Fusion Protein and Expression Vectors
The mtOFF Fusion Protein

One aspect of the present application relates to a fusion protein. The fusion protein (mtOFF) comprises a first moiety that targets the fusion protein to the mitochondrial inner membrane and a second moiety that comprises a light-activated proton pump, wherein the first moiety orients the proton pump in the direction to pump protons from the inner membrane space to the mitochondrial matrix (mtOFF direction). In some embodiments, the first moiety and the second moiety are linked directly to each other. In some embodiments, the first moiety and the second moiety are linked to each other through a peptide linker. In some embodiments, the fusion protein further comprises a marker for easy localization of the fusion protein within a cell or a mitochondrion. In some embodiments, the fusion protein is expressed within mitochondria to reduce the likelihood of immune responses to the fusion protein.

The first moiety of mtOFF targets the fusion protein to the mitochondria membrane and orient the fusion protein such that the proton pump pumps protons from the inner membrane space to the mitochondrial matrix. In some embodiments, the first moiety comprises a canonical mitochondrial targeting sequence and a generic transmembrane domain that orient the fusion protein such that the proton pump pumps protons from the inner membrane space to the mitochondrial matrix.

The mitochondrial targeting sequence can be any sequence capable of targeting the fusion protein to mitochondria membrane. Examples of mitochondrial targeting sequences include, but are not limited to, the mitochondrial targeting sequences of succinate dehydrogenase cytochrome b560 subunit, mitochondrial (SDHC), inner membrane mitochondrial proteins (IMMT), citrate synthase, aconitases, ATP synthase subunits (e.g. ATP5A), TOM70, NADH ubiquinone oxoreductases and mitochondrial ATP-ase inhibitors) and SDHD.

The transmembrane domain can be any transmembrane domain that is capable of orienting the fusion protein in the mitochondria membrane such that the proton pump pumps protons from the inner membrane space to the mitochondrial matrix. Any mitochondrial protein, or fragment thereof, with its N-terminus in the matrix that spans the inner membrane of mitochondria in an even number (e.g., 2, 4, 6, etc.) may be sufficient to target and orient mtOFF.

In some embodiments, the first moiety comprises an amino acid sequence from human SDHC (SEQ ID NO:1) and variants thereof, mouse SDHC (SEQ ID NO:4) and variants thereof, or rat SDHC (SEQ ID NO:7) and variants thereof that is capable of targeting and orienting the mtOFF construct in the mitochondria membrane.

In some embodiments, the first moiety comprises the first 138, 139, 140, 141, 142, 143 or 144 amino acids of the human SDHC or variants thereof. In some embodiments, the first moiety comprises the first 138, 139, 140, 141, 142, 143 or 144 amino acids of the mouse SDHC or variants thereof. In some embodiments, the first moiety comprises the first 138, 139, 140, 141, 142, 143 or 144 amino acids of the rat SDHC or variants thereof.

In some embodiments, the first moiety comprises the first 138 amino acids of human SDHC (SEQ ID NO:2) or variants thereof. In some embodiments, the first 144 amino acids of human SDHC (SEQ ID NO:3) or variants thereof.

In some embodiments, the first moiety comprises the first 138 amino acids of mouse SDHC (SEQ ID NO:5) or variants thereof. In some embodiments, the first moiety comprises the first 144 amino acids of mouse SDHC (SEQ ID NO:6) or variants thereof.

In some embodiments, the first moiety comprises the first 138 amino acids of rat SDHC (SEQ ID NO:8) or variants thereof. In some embodiments, the first moiety comprises the first 144 amino acids of mouse SDHC (SEQ ID NO:9) or variants thereof.

In some embodiments, the first moiety comprises a sequence that is 80%, 85%, 90%, 95%, 96%, 97%, 98%, 99% or 100% identical to the first 144 amino acids of human SDHC (SEQ ID NO:3).

In some embodiments, the first moiety comprises a sequence that is 80%, 85%, 90%, 95%, 96%, 97%, 98%, 99% or 100% identical to the first 144 amino acids of mouse SDHC (SEQ ID NO:6).

In some embodiments, the first moiety comprises a sequence that is 80%, 85%, 90%, 95%, 96%, 97%, 98%, 99% or 100% identical to the first 144 amino acids of rat SDHC (SEQ ID NO:9).

The second moiety of mtOFF may be any canonical light-activated proton pump. Examples of light-activated proton pump include, but are not limited to, Mac (Leptosphaeria maculans rhodopsin) and variants, such as eMac3.0; Arch (Halorubrum sodomense archaerhodopsin-3) and variants, such as ArchT, eArch3.0m and eArchT3.0; bacteriorhodopsin (bR) and the related delta rhodopsin (dR).

In some embodiments, the second moiety comprises an amino acid sequence that is 80%, 85%, 90%, 95%, 96%, 97%, 98%, 99% or 100% identical to from Mac (SEQ ID NO:10) and variants thereof.

In some embodiments, the second moiety comprises the amino acid sequence of SEQ ID NO:10.

In some embodiments, the first moiety is joined to the second moiety directly. In other embodiments, the first moiety is linked to the second moiety by a peptide linker. In some embodiments, the linker comprises hydrophilic residues. In some embodiments, the linker is the remainder resulting from the restriction cloning used to generate the fusion. In some embodiments, the linker is Pro-Ala-Gly.

In some embodiments, the fusion protein of the present application comprises the amino acid sequence of SEQ ID NO:11. In other embodiments, the fusion protein of the present application further comprise a fluorescent protein marker, such as GFP.

Polynucleotide Encoding the mtOFF Fusion Protein

Another aspect of the present application relates to a polynucleotide encoding the fusion protein of the present application. In some embodiments, the polynucleotide encodes a fusion protein (mtOFF) that comprises a first moiety that targets the fusion protein to the mitochondrial inner membrane and a second moiety that comprises a light-activated proton pump, wherein the first moiety orients the proton pump in the direction to pump protons from the inner membrane space to the mitochondrial matrix.

In certain embodiments, the polynucleotide encodes a fusion protein which is mammalian codon optimized. In some embodiments, the polynucleotide of the present application further comprises a coding sequence for an amino terminal signal peptide, which is removed from the mature protein. Since the signal peptide sequences can affect the levels of expression, the polynucleotides may encode any one of a variety of different N-terminal signal peptide sequences. It will be appreciated by those skilled in the art that the design of the polynucleotide of the present application can depend on such factors as the choice of the host cell to be transformed, the level of expression of protein desired, and the like.

In some embodiments, the polynucleotide comprises a sequence encoding the fusion protein of SEQ ID NO:11. In some embodiments, the polynucleotide comprises the sequence of SEQ ID NO:12.

### Expression Cassette

Another aspect of the application relates to an expression cassette that comprises one or more regulatory sequences operably linked to the coding sequence of the fusion protein of the present application. The fusion protein (mtOFF) comprises a first moiety that targets the fusion protein to the mitochondrial inner membrane and a second moiety that comprises a light-activated proton pump, wherein the first moiety orients the proton pump in the direction to pump protons from the inner membrane space to the mitochondrial matrix.

In some embodiments, the one or more regulatory sequences include a promoter and a ′3 UTR sequence. Preferred promoters are those capable of directing high-level expression in a target cell of interest. The promoters may include constitutive promoters (e.g., HCMV, SV40, elongation factor-1α (EF-1α)) or those exhibiting preferential expression in a particular cell type of interest. In some embodiments, a ubiquitous promoter such as a CMV promoter or a CMV-chicken beta-actin hybrid (CAG) promoter to control the expression of the fusion protein of the present application. In other embodiments, a tissue specific promoter, such as skin specific promotor, neuron specific promotor, muscle specific promoter and liver specific promoter, is used to control the expression of the fusion protein in a specific tissue. Tissue specific promoters are well known in the art.

In some embodiments, it is contemplated that certain advantages will be gained by positioning the coding sequence under the control of a recombinant, or heterologous, promoter. As used herein, a recombinant or heterologous promoter is intended to refer to a promoter that is not normally associated with a protein's gene in its natural environment. Such promoters may include promoters isolated from plant, insect, bacterial, viral, eukaryotic, fish, avian or mammalian cells. Naturally, it will be important to employ a promoter that effectively directs the expression of the DNA segment in the cell type chosen for expression. The use of promoter and cell type combinations for protein expression is generally known to those of skill in the art of molecular biology.

In some embodiments, the one or more regulatory sequences further comprise an enhancer. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the antibody producing cell.

In some embodiments, cell or tissue-specific transcriptional regulatory elements (TREs) can be incorporated into expression cassette to restrict expression to desired cell types. An expression vector may be designed to facilitate expression of the fusion proteins herein in one or more cell types.

In some embodiments, the expression cassette of the present application comprises a nucleotide sequence encoding the fusion protein of SEQ ID NO:11. In some embodiments, the expression cassette of the present application comprises the nucleotide sequence of SEQ ID NO: 12.

### Expression Vectors

Another aspect of the present application relates to an expression vector comprising the expression cassette of the present application. The expression cassette comprises (1) a polynucleotide encoding a fusion protein comprising a first moiety that targets the fusion protein to the mitochondrial inner membrane and a second moiety that comprises a light-activated proton pump, wherein the first moiety orients the proton pump in the direction to pump protons from the inner membrane space to the mitochondrial matrix; and (2) a regulatory sequence operably linked to the polynucleotide.

### Non-Viral Vectors

In some embodiments, the expression vector is a non-viral expression vector. In some embodiments, the non-viral expression vector is a plasmid capable of expressing the fusion protein of the present application in an in vitro and/or in vivo setting.

In some embodiments, non-viral expression vectors of the present application are introduced into cells or tissues by encapsulating the expression vectors in liposomes, microparticles, microcapsules, virus-like particles, or erythrocyte ghosts. Such compositions can be further linked by chemical conjugation to, for example, microbial translocation domains and/or targeting domains to facilitate targeted delivery and/or entry of nucleic acids into the nucleus of desired cells to promote gene expression. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

In some embodiments, non-viral expression vectors are introduced into the cells or tissues as naked DNA by direct injection or electroporation. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### Viral Vectors

In some embodiments, the expression vector of the present application is a viral expression vector. In certain embodiments, viral expression vectors may be engineered to target certain diseases and cell populations by using the targeting characteristics inherent to the virus vector or engineered into the virus vector. Specific cells may be “targeted” for delivery of polynucleotides, as well as expression.

In some embodiments, the viral expression vector is selected from the group consisting of retroviral vectors, lentivirus vectors, adenovirus vectors, adeno-associated virus (AAV) vectors and herpes virus vectors.

In some embodiments, the viral expression vector is a lentivirus vector. In some embodiments, the lentivirus vector is a non-primate lentivirus vector, such as equine infectious anemia virus (EIAV).

In some embodiments, the viral expression vector comprises a mitogenic T cell-activating transmembrane protein and/or a cytokine-based T cell-activating transmembrane protein in the viral envelope. In some embodiments, the viral expression vector is a lentiviral vector comprising a mitogenic T cell-activating transmembrane protein and/or a cytokine-based T cell-activating transmembrane protein in the viral envelope.

In some embodiments, the viral expression vector is a recombinant AAV vector (rAAV). rAAVs can spread throughout CNS tissue following direct administration into the cerebrospinal fluid (CSF), e.g., via intrathecal and/or intracerebral injection. In some embodiments, rAAVs (such as AAV-9 and AAV-10) cross the blood-brain-barrier and achieve wide-spread distribution throughout CNS tissue of a subject following intravenous administration. In some cases, intravascular (e.g., intravenous) administration facilitates the use of larger volumes than other forms of administration (e.g., intrathecal, intracerebral). Thus, large doses of rAAVs (e.g., up to 1015 rAAV genome copies (GC)/subject) can be delivered at one time by intravascular (e.g., intravenous) administration. Methods for intravascular administration are well known in the art and include, for example, use of a hypodermic needle, peripheral cannula, central venous line, etc.

Any suitable AAV serotype may be utilized for the recombinant AAV, including but not limited to AAV1, AAV2, AAV3, AAV4, AAV5, AAV6, AAV7, AAV8, AAV9, AAV10, AAV11, AAV12, and pseudotyped combinations thereof. Pseudotyped (or chimeric) AAV vectors include portions from more than one serotype, for example, a portion of the capsid from one AAV serotype may be fused to a second portion of a different AAV serotype capsid, resulting in a vector encoding a pseudotyped AAV2/AAV5 capsid. Alternatively, the pseudotyped AAV vector may contain a capsid from one AAV serotype in the background structure of another AAV serotype. For example, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8 and AAV2/9 serotype vectors. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV11, AAV12 or other known or as yet unknown AAV serotypes. These ITRs or other AAV components may be readily isolated from an AAV serotype using techniques available to those of skill in the art. In addition, AAV sequences may be isolated or obtained from academic, commercial, or public sources (e.g., the American Type Culture Collection, Manassas, Va.) or may be obtained through synthetic or other suitable means by reference to published sequences such as are available in the literature or in databases such as, e.g., GenBank, PubMed and the like.

It will be appreciated by those skilled in the art that the design of the expression vector of the present application can depend on such factors as the choice of the host cell to be transformed, the level of expression of protein desired, and the like.

mtOFF-Mitochondria

Another aspect of the present application relates to mitochondria (mtOFF-mitochondria) that comprise the mtOFF fusion protein of the present application in its inner membrane, wherein the mtOFF fusion protein is capable of pump protons from the inner membrane space to the mitochondrial matrix upon activation by light.

In some embodiments, the mtOFF-mitochondria are transferred directly into cells. In some embodiments, mtOFF-mitochondria are transferred into cells by first mixing them together with the cells followed by centrifugation. This method makes mitochondrial delivery possible into any cell type, and no additional incubation is required. The transfer efficiency remains high irrespective of the amounts of mitochondria used. In a specific embodiment, mtOFF-mitochondria are transferred into target cells via centrifugation at 1,500×g for 5 min without additional incubation. The exogenous mtOFF-mitochondria can be transferred regardless of cell type or species.

In a more specific embodiment, prior to mitochondrial transfer, recipient cells prelabelled with MITOTRACKER™ Green are harvested from culture flasks, and 1×10⁵ cells were transferred to a microcentrifuge tube. Cells were suspended in 100 μl of PBS and kept on ice for transfer. The mitochondrial suspension (in 10 μl of PBS) is added slowly to each tube of recipient cells suspended in 100 μl of PBS. The microcentrifuge tubes are centrifuged at 1,500×g for 5 min at 4° C. Cells are then rinsed twice with PBS and imaged or lysed for further testing.

In certain embodiments, mtOFF-mitochondria may be transferred to cells by use of Pep 1-conjugated mitochondria.

In certain embodiments, mtOFF-mitochondria may be transferred to cells by use of magnetic nanoparticles, such as by treating cultured cells with mitochondria labelled with anti-TOM22 magnetic beads and placing them on magnetic plates.

In certain embodiments, mtOFF-mitochondria may be transferred to cells by transferring mitochondria isolated from mesenchymal stem cells into cultured cancer cells. In specific embodiments, cancer cells are plated, mtOFF-mitochondria are added and cultures are centrifuged twice. Co-culture is then performed for 24 h to transfer mitochondria.

One of ordinary skill will understand that the means by which the mtOFF-mitochondria are transferred into target cells whether in vivo, ex vivo, or in vitro is not limiting on the scope of the application.

mtOFF Cells

Another aspect of the application relates to cells (mtOFF-cells) comprising mitochondria (mtOFF-mitochondria) comprising a mtOFF fusion protein. The mtOFF fusion protein comprises a first moiety that targets the fusion protein to the mitochondrial inner membrane and a second moiety that comprises a light-activated proton pump, wherein the first moiety orients the proton pump in the direction to pump protons from the inner membrane space to the mitochondrial matrix.

The cells types that may be targeted for use of mtOFF-mitochondria include, but are not limited to, T-cells, neurons, retinal cells, stem cells, hematopoietic stem cells, induced pluripotent stem cells, blood cells, epithelial cells, muscle cells, sperms and eggs, interneurons, glial cells, fat cells, hair follicles, keratinocytes.

In some embodiments, a mtOFF cell is generated by introducing an expression vector of the present application into a target cell with any conventional method, such as by naked DNA technique, cationic lipid-mediated transfection, polymer-mediated transfection, peptide-mediated transfection, virus-mediated infection, physical or chemical agents or treatments, electroporation, etc.

In some embodiments, a mtOFF cell is generated by transferring one or more mtOFF mitochondria into a target cell.

Administration of mtOFF Expression Vectors

Any suitable route or mode of administration can be employed for providing a subject with a therapeutically or prophylactically effective dose of the mtOFF expression vector. Exemplary routes or modes of administration include parenteral (e.g., intravenous, intraarterial, intramuscular, subcutaneous, intratumoral), topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

### Methods of Use

A further aspect of the present application relates to a method of treating, or ameliorating symptoms of, diseases or conditions that are characterized by raised mitochondrial activity or mitochondrial dysfunction due to increased PMF or abnormally high ATP/ADP, NADH/NAD+, or FADH2/FAD ratios in a target cell population of a subject. In some embodiments, the method comprises the steps of introducing mtOFF fusion proteins into the mitochondria of the cell population to generate mtOFF cells, and exposing the mtOFF cells to light to activate the proton pump to increase mitochondrial activity, wherein the mtOFF fusion protein comprises a first moiety that targets the fusion protein to the mitochondrial inner membrane and a second moiety that comprises a light-activated proton pump, wherein the first moiety orients the proton pump in the direction to pump protons from the inner membrane space to the mitochondrial matrix.

In some embodiments, the method further comprises administering to the subject a second therapeutic agent. The second therapeutic agent can be administered to the subject before, after, or concurrently with the mtOFF fusion protein.

Diseases and conditions that are characterized by raised mitochondrial activity include, but are not limited to, tumors, cancers, inflammatory disorders and immune disorders.

Diseases and conditions that are characterized by raised mitochondrial activity also include disorders of mitochondrial metabolism, such as psoriasis, muscle hypertonicity disease and/or muscle recovery following stress/sports induced muscle tension, and treating fungal infection by targeting mtOFF specifically to fungal cells.

Diseases and conditions that may be related mitochondrial dysfunction due to increased PMF include, but are not limited to, retinal degeneration, and seizures.

The mtOFF fusion protein may be introduced into mitochondria of the target cell population by introducing a non-viral expression vector capable of expressing the mtOFF protein into the target cells. Alternatively, the mtOFF fusion protein may be introduced into the target cell population by infecting the target cells with a viral vector capable of expressing the mtOFF protein in the target cells. The location and timing of the mtOFF expression may vary depending on the target cell population and the diseases or conditions to be treated by the method.

In some embodiments, mtOFF cells are generated in vitro by transfecting or infecting cultured cells with mtOFF expression vectors. The culture mtOFF cells are then transferred into the subject for treating, or ameliorating symptoms of, diseases or conditions that are characterized by raised mitochondrial activity, abnormally high ATP levels or mitochondrial dysfunction. In some embodiments, the cultured cells are cells autologous to the subject. In some embodiments, the cultured cells are cells allogeneic to the subject. Examples of such cells include, but are not limited to, T cells, natural killer cells, stem cells, hematopoietic stem cells, blood cells, neurons, interneurons, muscle cells, glial cells, fat cells, epithelial cells, hair follicles and karatinocytes.

After expression of the mtOFF protein, the resulting mtOFF cells will be exposed to light to activate the proton pump of the mtOFF protein. The wave length of the light depends on the characteristics of the light-sensitive proton pump on the mtOFF fusion protein. In some embodiments, light with a wave length in the range of 350-750 nm, preferably 450-650 nm is used for the activation of Arch and variant. In some embodiments, light with a wave length in the range of 325-725 nm, preferably 425-625 nm is used for the activation of Mac and variant. In some embodiments, light with a wave length in the range of 300-750 nm, preferably 400-650 nm is used for the activation of bacteriorhodopsin and variants, as well as delta rhodopsin and variants. The light intensity and length of light-exposure may be adjusted to establish protonmotive force (PMF) in mitochondria of the mtOFF cells to achieve desired therapeutic effect. In some embodiments, the activation light is provided by an LED system implanted in the subject. In some embodiments, the activation light is provided through an optical fiber.

The mtOFF constructs may be adapted for expression in plants for myriad applications. mtOFF manipulates mitochondrial protonmotive force and metabolism broadly in living organisms. In plants, mitochondria provide energy similarly to their role in metazoan organisms. Using mtOFF to manipulate protonmotive force in plants can result in applications to control the growth rate of plants, crop yields, quality of crop yields, and disease or parasite resistance. Metabolism is a fundamentally important parameter for each of these aspects of plant life. Plants naturally use visible light for photosynthesis and are an evident system to apply mtOFF. The absorbance spectrum of mtOFF is not widely used in plants and would allow the construct to control plant energy and reactive oxygen species production. Of note, unlike respiration, mtOFF activity does not require oxygen or metabolic substrates and can reduce reactive oxygen species production, a byproduct of metabolism.

Hair forms a protective barrier and has roles in social interactions. Hair generation requires the activation of hair follicle stem cells through an energy intensive process and aging can alter this, resulting in the graying and thinning of hair. Androgenetic alopecia (male/female pattern hair loss) is a common form of hair loss and can result from stress, environmental insults or aging. There is no cure for baldness. Current therapies are limited by incomplete efficacy and serious adverse effects. mtOFF can be used to directly alter metabolism to reverse hair thinning and loss associated with aging. Stress-induced hair graying occurs through reversible changes in mitochondrial function (PMID:34155974). The activation of mtOFF can be used to suppress dysfunctional mitochondrial dysfunction. Although mitochondria are directly implicated in hair follicle regeneration, current approaches to selectively activate mitochondrial metabolism are limited.

Mitochondria supply energy for cellular activity. Mitochondria are much like batteries and use metabolic substrates to generate a protonmotive force, which is a charge separation that is used to do work. As humans age, mitochondria become dysfunctional and the machinery that produces the protonmotive force becomes damaged resulting in impaired energy production. Since mitochondria are important for survival, dysfunction is implicated in numerous pathologies, such as psoriasis and skin inflammation. Thus, an ideal approach would selectively silence dysfunctional mitochondria in the skin through a noninvasive approach amenable to home use.

Mitochondria are the metabolic hub of the cell and can signal energy status to the cell through signaling cascades. The activity of mtOFF can activate AMPK signaling activity. AMPK is a master regulator of a cells metabolic status and is activated under conditions of low energy or starvation. AMPK activity is suppressed under conditions of abundant energy sources or plentiful food. Using mtOFF it is possible to manipulate C. elegans feeding behavior. For example, the activation of mtOFF made worms behave as if they were starved despite the presence of food. These studies also showed that this process is mediated through neurons only. Based on these findings, mtOFF can be used to control hunger.

In certain embodiments, such as in humans or mice (and other species that produce ATR endogenously), supplementation using ATR is not performed; in other embodiments, such as C. elegans, ATR supplementation is used in conjunction with mtOFF.

In certain embodiments, the target cells are selected from the group consisting of stem cells, epithelial cells, retinal cells, T-cells, and hematopoietic stem cells and blood cells.

In some embodiments, the target cells are retinal cells and the mtOFF fusion proteins are introduced into the retinal cells with AAV mediated gene transfer to the eye.

In some embodiments, the target cells are blood cells. In some embodiments, the blood cells are isolated from the subject, infected or transfected in vitro with a mtOFF expression vector, and then transferred back to the subject.

In some embodiments, the target cells are stem cells. In some embodiments, the stem cells are isolated from the subject, differentiated in vitro, infected or transfected with a mtOFF expression vector in vitro, and then transferred back to the subject.

### Method of Use Relating to Hypoxia

In some embodiments, the present application provides a method for modulating hypoxia signaling in a subject. The method comprises the steps of: administering to the subject an effective amount of an expression vector comprising a polynucleotide encoding a mtOFF fusion protein, expressing the mtOFF fusion protein in a group of target cells to generate mtOFF cells; and exposing the mtOFF cells to light for a desired period of time to modulate hypoxia signaling.

In some embodiments, the present application provides a method for improving hypoxia resistance in a subject. The method comprises the steps of: administering to the subject an effective amount of an expression vector comprising a polynucleotide encoding a mtOFF fusion protein, expressing the mtOFF fusion protein in a group of target cells to generate mtOFF cells; and exposing the mtOFF cells to light for a desired period of time to improve hypoxia resistance.

In some embodiments, the present application provides a method for preventing/ameliorating ischemia reperfusion injury in a subject. The method comprises the steps of: administering to the subject an effective amount of an expression vector comprising a polynucleotide encoding a mtOFF fusion protein, expressing the mtOFF fusion protein in a group of target cells to generate mtOFF cells; and exposing the mtOFF cells to light for a desired period of time to prevent or ameliorate ischemia reperfusion injury. In some embodiments, the ischemia reperfusion injury is caused by heart attack or stroke. In some embodiments, the target cells are cardiomyocytes, smooth muscle cells, cardiac neurons and/or endothelial cells. In some embodiments, the expression vectors are administered prior to the start of ischemia. In some embodiments, the expression vectors are administered during ischemia. In some embodiments, the expression vectors are administered after ischemia.

In some embodiments, the present application provides a method preventing/ameliorating ischemia reperfusion injury in a subject in the context of elective surgery or organ transplant, transfection or other suitable mechanisms may introduce the expression vector to the subject as a way of preparing the subject to modulate or improve hypoxia resistance. In certain embodiments, mtOFF fusion proteins are introduced into cardiac muscle during an elective cardiac surgery when the heart is stopped or put on bypass. In certain embodiments, mtOFF fusion proteins are introduced into transplanted tissues/organ to prevent or ameliorate ischemia reperfusion injury to the transplanted tissues/organ.

### Method of Use Relating to Neurodegenerative Diseases

In some embodiments, the present application relates to a method of treating or ameliorating symptoms of neurodegenerative diseases in a subject, comprising the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix; exposing the target cells to light to activate the proton pump to decrease the protonmotive force (PMF) across the mitochondrial inner membrane, wherein decreased PMF in the mitochondria of the target cells prevents development of symptoms, or ameliorates existing symptoms, of neurodegenerative diseases.

Without being bound by theory, in the cell, there are many mitochondrion and they are constantly being made (mitochondrial biogenesis) and degraded (mitochondrial autophagy, aka mitophagy). Over time individual mitochondria can accumulate damage, become dysfunctional and are targeted for degradation via autophagy—this acts as a quality control mechanism. Maintaining a healthy/functional population is critical for cell survival especially for energy demanding tissues such as cardiomyocytes or neurons. Mitochondrial dysfunction is the hallmark of many neurodegenerative diseases. For some of these disease (e.g., Parkinson's, Alzheimer's) the activation of autophagy is protective. This is also true for heart attacks. mtOFF activation would remove damage-causing dysfunctional mitochondrion by signaling for their destruction via autophagy. This would then cause mito-biogenesis of new functional mitochondrion. Thus, the removal of damaged mitochondria (via mtOFF activation of autophagy) can ameliorate the disease.

In some embodiments, PMF in the mitochondria of the target cells is decreased to an extent that results in mitochondria autophagy. In some embodiments, the light-activated proton pump in the second moiety is a Mac proton pump.

In some embodiments, the target cells are neuronal cells. In some embodiments, the fusion protein is expressed in the target cells by infecting the target cells with a viral vector capable of expressing the fusion protein in the target cells.

### Method of Use Relating to Cell Resistance to Stress

In some embodiments, the present application relates to a method of enhancing cell resistance to stress in a subject, comprising the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix, wherein the first moiety comprises a targeting/orienting sequence from SDHC; exposing the target cells to light to activate the proton pump to decrease the protonmotive force (PMF) across the mitochondrial inner membrane, wherein decreased PMF in the mitochondria of the target cells enhances the target cells' resistance to stress.

In some embodiments, PMF in the mitochondria of the target cells is decreased to an extent that results in mitochondria autophagy. In some embodiments, the light-activated proton pump in the second moiety is a Mac proton pump.

### Method of Use Relating to Cancer Treatment

In some embodiments, the present application relates to a method of treating cancer in a subject suffering from cancer, comprising the steps of: expressing a fusion protein in target cancer cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix; exposing target cancer cells to light to activate the proton pump to decrease the protonmotive force (PMF) across the mitochondrial inner membrane, wherein decreased PMF in the mitochondria of the target cancer cells inhibits cancer cell growth in the subject.

In some embodiments, PMF in the mitochondria of the target cancer cells is decreased to an extent that results in mitochondria autophagy.

Method of Use for Treating Metabolic Disorders and/or Conditions

In some embodiments, the present application relates to a method for treating, preventing or ameliorating symptoms of metabolic disorders/conditions caused by mitochondrial dysfunction (e.g., abnormal high levels ATP/NADH/FADH2) in a subject. The method comprises the steps of: expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix, wherein the first moiety comprises a targeting/orienting sequence from SDHC; exposing the target cells to light to activate the proton pump to decrease the protonmotive force (PMF) across the mitochondrial inner membrane, wherein decreased PMF in the mitochondria of the target cells prevents development symptoms, or ameliorating existing symptoms, of the metabolic disorder.

Examples of metabolic disorders/conditions caused by mitochondrial dysfunction include, but are not limited to, skin inflammation, such as psoriasis.

In some embodiments, the PMF in mitochondria of the target cells is decreased to an extent that results in mitochondria autophagy.

In some embodiments, the mtOFF fusion protein is used to reduce metabolic activity in fungal cells that may cause fungal infection (e.g., nail infection).

In some embodiments, the mtOFF fusion protein is used to treat muscles hypertonicity condition/easing the symptoms.

In some embodiments, the mtOFF fusion protein is used to treat strained muscles in order to accelerate recovery for athletes.

In some embodiments, the mtOFF fusion protein is used for organ maintenance during organ transplant shipping/operation

In some embodiments, the mtOFF fusion protein is used as a senolytic to selectively induce death of senescent cells.

In some embodiments, the mtOFF fusion protein, the mtOFF expression vector and/or the mtOFF cells may be formulated in a topical treatment composition. In some embodiments, the the mtOFF fusion protein, the mtOFF expression vector and/or the mtOFF cells may be co-formulated with one or more skincare ingredients. In some embodiments, the one or more skincare ingredients may be small molecule compounds, polymers, peptides or cells. In some embodiments, the one or more skincare ingredients are selected from the group of alpha-hydroxy acids, polyhydroxy acids, beta-hydroxy acid (salicylic acid), hydroquinone, kojic acid, retinoids, L-ascorbic acid, hyaluronic acid, copper peptide, alpha-lipoic acid, and DMAE (dimethylaminoethanol),

In some embodiments, the topical treatment composition is formulated for application to human skin. More specifically, the formulation can be configured to penetrate topically from the epidermis to the dermis. In some embodiments, the formulation can be configured to penetrate topically through the epidermis and dermis layers. In some embodiments, the formulation can be configured to penetrate topically through the epidermis layer and have low penetration into the dermis layer. Often, the penetration of a component in a formulation may be assessed using various permeation studies, including but not limited to those using a Franz diffusion cell. In some embodiments, the formulation comprises a carrier, a microsphere, a liposome, or a micelle in order to carry the mtOFF fusion protein, the mtOFF expression vector and/or the mtOFF cells and control the release time and/or penetration depth of the the mtOFF fusion protein, the mtOFF expression vector and/or the mtOFF cells through the skin. In some cases, a formulation herein is a cream, an ointment, a gel, a liquid, an oil, a powder, a lotion, a serum, an emulsion, a moisturizer, a foam, a face mask, a mousse, an aerosol, a spray, a cleanser, a toner, a topical patch, a hydrogel patch, or a shampoo.

In some embodiments, the formulation further comprises a therapeutic, nutraceutical, or cosmetic excipient. In some embodiments, the administering comprises applying the formulation to a portion of the skin of the subject. In some embodiments, the formulation extends a lifespan of a plurality of cells of the subject, induces SIRT6 expression in a plurality of cells of the subject, increases cell renewal rates in a plurality of cells of the subject, promotes apoptosis in a plurality of cells of the subject, promotes DNA repair in a plurality of cells of the subject, increases collagen production in a plurality of cells of the subject, increases hyaluronic synthase production in a plurality of cells of the subject, decreases ATRX nuclear foci accumulation in a plurality of cells of the subject, decreases p16 expression in a plurality of cells of the subject, decreases senescence associated beta-galactosidase production in a plurality of cells of the subject, decreases IL8 expression in a plurality of cells of the subject, decreases MMP1 expression in a plurality of cells of the subject, increases BLM expression in a plurality of cells of the subject, and/or prevents UV-induced DNA damage in a plurality of cells of the subject.

In particular embodiments, a mtOFF fusion protein, mtOFF expression vector and/or a mtOFF cell composition can be formulated for topical application. For example, the composition may be formulated for application onto skin. In some embodiments, the composition is configured as a topical supplement. Formulations such as those for topical application can be a cream, an ointment, a gel, a liquid, a powder, a lotion, a serum, an emulsion, a moisturizer, a foam, a face mask, a mousse, an aerosol, a spray, a cleanser, a toner, a topical patch, a hydrogel patch, or a shampoo. mtOFF fusion protein, mtOFF expression vector and/or mtOFF cells applied topically can be applied to an affected area, to an area which may become affected in the future, a portion of the subject, or substantially the entire subject. In some cases, a topical treatment can be applied with a buffer, another topical treatment, a cream, or a moisturizer.

A composition, such as for topical application, can be formulated as a cosmetic composition. Examples of cosmetic compositions can include makeup, foundation, sunscreen, after sun lotion, and skin care products, including anti-aging skin care products. In some cases, makeup compositions can leave color on the face, and can include foundation, bronzer, mascara, concealer, eye liner, brow color, eye shadow, blusher, lip color, powder, a solid emulsion compact, or other makeup items. In some cases, skin care products can be those used to treat or care for, or somehow moisturize, improve, accelerate renewal, protect, prevent damage, or clean the skin. A skin-care product can be applied as a cream, a topical patch, a hydrogel patch, a transdermal patch, an ointment, a gel, a liquid, a powder, a lotion, a serum, an emulsion, an oil, a clay, a moisturizer, a foam, a face mask, a mousse, an aerosol, a spray, a cleanser, a toner, or a shampoo. In some cases, skin-care products can be in the form of an adhesive, a bandage, exfoliant, a toothpaste, a moisturizer, a lotion, a primer, a lipstick, a lip balm, an anhydrous occlusive moisturizer, an antiperspirant, a deodorant, a personal cleansing product, an occlusive drug delivery patch, a nail polish, a powder, a tissue, a wipe, a hair conditioner, or a shaving cream.

In some cases, a composition can comprise a skin conditioning agent (e.g., a humectant, exfoliant, emollient, or hydrator). A humectant can be for moisturizing, reducing scaling, or stimulating removal of built-up scale from the skin. An exfoliant can be for the removal of old skin cells from the surface, and can be a physical exfoliant or a chemical exfoliant. An emollient can be a preparation or ingredient which can soften dry, rough, or flakey skin. A hydrator can be for moisturizing, reducing scaling, or stimulating removal of built-up scale from the skin. In some cases, emollient is an agent that prevents water loss and has a softening and soothing effect on skin. In some embodiments, emollients may comprise at least one of plant oils, mineral oil, shea butter, cocoa butter, petrolatum, fatty acids (animal oils, including emu, mink, and lanolin), triglycerides, benzoates, myristates, palmitates, stearates, glycolipids, phospholipids, squalene, glycerin, rose hip oil, andiroba oil, grape seed oil, avocado oil, plum seed oil, pracaxi oil, Calycophyllum spruceanum oil, almond oil, argan oil, caprylic/capric triglyceride, jojoba butter, jojoba oil, Spectrastat G2, ceramide, and algae extract. In some cases, the composition comprises a skin hydrating agent, also referred to as a skin hydrator. In some cases, the skin hydrating agent include but are not limited to glycerin, squalene, sorbitol, hyaluronic acid, hyaluronic acid derivatives, sodium hyaluronate, sodium hyaluronate crosspolymer, niacinamide, glycoproteins, pyrrolidone carboxylic acid (PCA), lysine HCl, allantoin and algae extract. In some embodiments, the composition comprises at least 1%, 2%, 3%, 4%, 5%, 6%, 7%, 8%, 9%, or 10% skin conditioning agent. In some embodiments, the composition comprises about 1% to about 70%, about 1% to about 60%, about 1% to about 50%, about 5% to about 50%, about 5% to 45%, or about 5% to 40% skin conditioning agent.

A composition can comprise a shine control agent, which can improve or regulate the shiny appearance of skin. Shine control agents can be porous in nature. Such agents can provide a reservoir to absorb excess moisture to reduce the appearance of shine. Shine control agents can be silicas, magnesium aluminum silicates, talc, sericite and various organic copolymers. Particularly effective shine control agents can include silicates or carbonates that are formed by reaction of a carbonate or silicate with the alkali (IA) metals, alkaline earth (IA) metals, or transition metals, and silicas (silicon dioxide). Preferred shine control agents are selected from the group consisting of calcium silicates, amorphous silicas, calcium carbonates, magnesium carbonates, zinc carbonates, bentonite clay, and combinations thereof.

A composition can comprise a film forming agent, which can aid film substantivity and adhesion to the skin. A film forming agent can improve long wear and non-transfer performance of a composition. Film forming agents can be water soluble, water insoluble, or water dispersing. Film forming agents can be 1) organic silicone resins, fluorinated silicone resins, copolymers of organic silicone resins, trimethylsiloxysilicate, GE's copolymers of silicone resins, SF1318 (silicone resin and an organic ester of isostearic acid copolymer) and CF1301 (silicone resin and alpha methyl styrene copolymer), Dow Corning's pressure sensitive adhesives copolymers of silicone resins and various PDMS's (BIO-PSA series); and 2) acrylic and methacrylic polymers and resins, silicone-acrylate type copolymers and fluorinated versions of, including silicones plus polymer from 3M, KP545 from Shin-Etsu, alkyl-acrylate copolymers, KP 561 and 562 from Shin-Etsu; 3) decene/butene copolymer from Collaborative Labs; 4) polyvinyl based materials, PVP, PVPNA, including Antaron/Ganex from ISP (PVP/Triacontene copolymer), Luviskol materials from BASF; polyurethanes, the Polyderm series from Alzo including but not limited to Polyderm PE/PA, Polyderm PPI-SI-WS, Polyderm PPI-GH, Luviset P.U.R. from BASF; 6) polyquaternium materials, Luviquat series from BASF; 7) acrylates copolymers and acrylates/acrylamide copolymers, Luvimer and Ultrahold series, both available from BASF; 8) styrene based materials; and 9) chitosan and chitosan based materials including cellulose and cellulose-based materials.

A composition can comprise a thickening agent or an emulsifying agent. A thickening agent may be used to increase the viscosity of liquid base materials to be used in a cosmetic composition. The selection of a particular thickening agent can depend on a type of composition desired (e.g., gel, cream, lotion, or wax based), the desired rheology, the liquid base material used, and other materials to be used in the composition. Examples of thickening agent or an emulsifying agent can include waxy materials such as candelilla, carnauba waxes, beeswax, spermaceti, carnauba, baysberry, montan, ozokerite, ceresin, paraffin, synthetic waxes such as Fisher-Tropsch waxes, silicone waxes (DC 2503 from Dow Corning), microcrystalline waxes and the like; soaps, such as the sodium and potassium salts of higher fatty acids, acids having from 12 to 22 carbon atoms; amides of higher fatty acids; higher fatty acid amides of alkylolamines; dibenzaldehyde-monosorbitol acetals; alkali metal and alkaline earth metal salts of the acetates, propionates and lactates; and mixtures thereof. Also useful are polymeric materials such as, locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose, cellulose derivatives, cellulose ethers hydroxyethyl cellulose, methyl cellulose, hydroxypropyl cellulose, carboxymethyl cellulose, hydroxy propylmethyl cellulose, polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, poly(acrylic acid), carbomers, polymethacrylic acid polymers, polyvinyl acetate polymers, polyvinyl chloride polymers, polyvinylidene chloride polymers and the like. Inorganic thickeners may also be used such as aluminum silicates, such as, for example, bentonites, or a mixture of polyethylene glycol and polyethylene glycol stearate or distearate. An emulsifier may be used to help keep hydrophilic and hydrophobic ingredients from separating in an emulsion. In some cases, emulsifiers include but are not limited to Olivem, Oliwax LC, polysorbates, laureth-4, and potassium cetyl sulfate.

A cosmetic composition can provide a temporary change in an appearance or can provide a long-term change in an appearance. In some cases, a cosmetic composition can be formulated to provide a short-term change in an appearance (e.g., color deposition or plumping of skin) as well as a long-term change in appearance (e.g., reduction in spots, appearance of fine lines, appearance of wrinkles, or other features which can affect appearance).

A composition can comprise an additive that has an additive or synergistic effect when applied with the mtOFF fusion protein, the mtOFF expression vector and/or the mtOFF cells as disclosed herein. For example, a composition comprising the mtOFF fusion protein, the mtOFF expression vector and/or the mtOFF cells and an additive can have a greater effect on senescence, and age-related disease or condition, or an age-associated disorder (e.g., delay the onset of, reduce the occurrence of, or ameliorate one or more symptoms) than the individual effect of the additive, the polypeptide, or the sum of the individual effects of the additive and the mtOFF fusion protein, the mtOFF expression vector and/or the mtOFF cells. Additives can be a polypeptide, a glycosaminoglycan, a carbohydrate, a polyphenol, a protein, a lipid, a plant aqueous or oil extract, a nucleic acid, an antibody, a small molecule, a vitamin, a humectant, an emollient, or another suitable additive. In some embodiments, the composition comprises a UV blocker. In some embodiments, the UV blocker may include but is not limited to aminobenzoic acid, avobenzone, cinoxate, dioxybenzone, homosalate, meradimate, octocrylene, octinoxate, octisalate, oxybenzone, padimate O, ensulizole, sulisobenzone, titanium dioxide, trolamine salicylate, and zinc oxide.

Often the methods, systems, and compositions provided herein comprise a vitamin. In some instances, the vitamin provides skin soothing, skin restoring, skin replenishing, and/or hydrating effects. In some instances, the vitamin provides antioxidant effects. In some instances, the vitamin acts as an emollient. In some instances, the vitamin improves the appearance of enlarged pores, uneven skin tone, fine lines, dullness, and/or a weakened skin surface. In some instances, the vitamin is vitamin A, vitamin D, vitamin E, vitamin F, vitamin K, vitamin B1 (thiamine), vitamin B2 (riboflavin), vitamin B3 (niacin), vitamin B5 (pantothenic acid), vitamin B7 (biotin), vitamin B6, vitamin B12 (cyanocobalamin), vitamin B9, folic acid, niacinamide, and mixtures thereof. In some instances, the composition comprises a derivative of a vitamin. In some instances, a derivative of a vitamin is used to improve stability of the vitamin in the composition and/or compatibility of the vitamin derivative with other ingredients in the composition. In some instances, the composition comprises vitamin B3 or its derivative and vitamin E or its derivative. In some instances, the composition comprises niacinamide and vitamin E or its derivative. In some instances, the composition comprises vitamin C or its derivative, vitamin B3 or its derivative, and vitamin E or its derivative. In some embodiments, the composition comprises at least 0.01%, 0.05%, 0.5%, 1%, 2%, 3%, 4%, 5%, 6%, 7%, 8%, 9%, or 10% vitamin. In some embodiments, the composition comprises about 0.1% to about 10%, about 0.1% to about 5%, about 0.5% to about 10%, about 0.5% to about 5%, about 1% to 10%, or about 1% to 5% vitamin.

If intended for transdermal administration, the composition may include a transdermal patch or iontophoresis device. In some cases, biodegradable microspheres (e.g., polylactic acid) may also be employed as carriers for a composition. In some cases, the transdermal patch is prepared to deliver the formulation to the epidermal layer of the skin. In some cases, the transdermal patch is prepared to deliver the formulation to the epidermal and dermal layers of the skin. In some cases, the formulation is prepared as to be minimally delivered systemically in the subject or is not intended to be delivered directly into the bloodstream of the subject.

In some cases, the age-related disease or condition or age-associated disorder can be a disease, condition, or disorder affecting the skin, such as a skin disorder or a dermatosis, which can comprise wrinkles, lines, dryness, itchiness, spots, age spots, bedsores, ulcers, cancer, dyspigmentation, infection (e.g., fungal infection), or a reduction in a skin property such as clarity, texture, elasticity, color, tone, pliability, firmness, tightness, smoothness, thickness, radiance, luminescence, hydration, water retention, skin barrier, evenness, laxity, or oiliness, or other dermatoses. In some instances, the age-related disease or condition or age-associated disorder is hyperpigmentation of the skin. In some instances, the hyperpigmentation disorder is melasma, age spots, lentigines, and/or progressive pigmentary purpura. In some instances, the hyperpigmentation is a result of sun damage, inflammation, hormone changes, or skin injuries. In some instances, the hyperpigmentation occurs after a cosmetic procedure, including but not limited to a laser treatment, a light treatment, or a chemical peel; administration of an antibiotic, an oral contraceptive, or a photosensitizing drug; or application of a topical agent. In some instances, the hyperpigmentation is a result of excess production of melanin.

In some instances, treatment of the age-related disease or condition or age-associated disorder with the methods, systems and compositions disclosed herein results in lightening, increasing luminescence, brightening, evening, smoothing and/or firming of the skin's appearance. In some instances, treatment with the methods, systems, and compositions disclosed herein improves the epidermal barrier, skin hydration level, skin water retention, appearance of wrinkles, smoothness, firmness, elasticity, appearance of radiance and luminosity, and/or improves or maintains the ceramide level in the skin. In some instances, the effect of treatment with the methods, systems, and compositions disclosed herein is assessed by measuring skin moisture content, trans-epidermal water loss (TEWL), dermal thickness and echogenicity, intracutaneous analysis, skin viscoelastic properties, or skin surface profile. In some instances, the effect of treatment with the methods, systems, and compositions disclosed herein assesses for reduction in appearance of lines/wrinkles, appearance of skin tone (evenness), appearance of pores, appearance of texture/smoothness, firmness (visual), elasticity (tactile), epidermal barrier, skin roughness, skin hyperpigmentation, or overall appearance. In some instances, the effect of treatment with the methods, systems, and compositions disclosed herein is measured using an instrument, including but not limited to a corneometer for measuring skin moisture content/hydration, a VapoMeter for measuring the trans-epidermal water loss (TEWL), an ultrasound measuring dermal thickness (density) and echogenicity, a non-invasive optical skin imaging instrument for measuring skin evenness and chromophore mapping, a cutometer using suction for measuring viscoelastic properties of the skin (firmness and elasticity), skin profilometry, multi-spectral analysis, and colorimetry for measuring skin surface profile, lines, and wrinkles.

In some instances, the methods, systems, and compositions provided herein may reduce hyperpigmentation of the skin. In some instances, hyperpigmentation is associated with excess production of melanin. In some instances, the methods, systems, and compositions provided herein reduces the excess production of melanin. In some instances, the methods, systems, and compositions provided herein reduce the presence of melanin pigment in the skin. In some instances, the methods, systems, and compositions provided herein reduce the expression levels of proteins involved in melanogenesis, including tyrosinase, melanocyte inducing transcription factor (MITF) and dopachrome tautomerase (DCT), by the cells in the treated skin. In some instances, the methods, systems, and compositions provided herein result in reduction of tyrosinase activity, reduction of the expression or activation of tyrosinase, scavenging of the intermediate products of melanin synthesis, reducing the transfer of melanosomes to keratinocytes, reduction of existing melanin content, or reduction in melanocyte activity or viability.

An age-related disease or condition or age-associated disorder can be caused by UV damage, DNA damage, ATRX foci accumulation in cell nuclei, increased p16 expression, increased senescence-associated beta-galactosidase activity, accumulation of senescent cells in the tissue, increased SASP production, chemically induced senescence, chronological aging, decreased hyaluronic acid production, decreased expression of sirtuin 6, altered insulin-like growth factor-1 (IGF-I) pathway signaling, increased production of matrix metallopeptidase 1 (MMP1), thin epidermal layer of the skin, or genetic variants. In some instances, the age-related disease or condition or age-associated disorder is initiated or exacerbated by a therapeutic regimen, for example, a side effect of a therapeutic drug. An age-related disease or condition or age-associated disorder can affect the health or appearance of skin directly or indirectly. Topical application of a mtOFF fusion protein, a mtOFF expression vector and/or mtOFF cells can improve the health or appearance of skin in some such cases.

An age-related disease or condition or age-associated disorder can comprise a cell proliferative disorder. A cell-proliferative disorder can affect the health or appearance of the skin. In some cases, a treatment administered for a cell-proliferative disorder, such as chemotherapy or radiation can affect the health or appearance of the skin. Topical application of a mtOFF fusion protein, a mtOFF expression vector and/or mtOFF cells can improve the health or appearance of skin in some such cases.

Also provided herein are methods for treating the skin of a subject comprising administering to a subject a composition that can promote a decrease in a number of senescent cells in a tissue or organism, inducing a pro-apoptotic state in the treated cells, inducing SIRT6 expression, preventing DNA-induced senescence, and/or enhancing DNA repair capacity. In some cases, a skin disease such as a dermatological disease or condition can comprise skin sagging or wrinkling, accumulation of senescent cells in the tissue, decreased epidermal thickness, decreased collagen production, increased MMP-1 production, decreased DNA repair capacity, decreased SIRT6 expression, skin disorganization, a thin epidermal layer of the skin, inflammation, a senescence-associated secretory phenotype, or stem cell exhaustion of the skin.

### Pharmaceutical Compositions

A pharmaceutical composition comprising a mtOFF expression vector in accordance with the present disclosure may be formulated in any pharmaceutically acceptable carrier(s) or excipient(s).

In some embodiments, mtOFF expression vectors can be incorporated into a pharmaceutical composition suitable for parenteral administration. In some embodiments, the pharmaceutical composition comprises a buffer. Suitable buffers include but are not limited to, sodium succinate, sodium citrate, sodium phosphate or potassium phosphate. In some embodiments, the pharmaceutical composition comprises sodium chloride at a concentration of 0-300 mM (optimally 150 mM for a liquid dosage form).

In some embodiments, the pharmaceutical composition is in a lyophilized dosage form and comprise a cryoprotectant. Examples of cryoprotectants include, but are not limited to, sucrose (optimally 0.5-1.0%), trehalose and lactose. In some embodiments, the pharmaceutical composition further comprises a bulking agent. Examples of bulking agents include, but are not limited to, mannitol, glycine and arginine.

Therapeutic preparations can be lyophilized and stored as sterile powders, preferably under vacuum, and then reconstituted in bacteriostatic water (containing, for example, benzyl alcohol preservative) or in sterile water prior to injection. Pharmaceutical composition may be formulated for parenteral administration by injection e.g., by bolus injection or continuous infusion.

The pharmaceutical forms suitable for injectable use include sterile aqueous solutions or dispersions and sterile powders for the extemporaneous preparation of sterile injectable solutions or dispersions. The form should be sterile and fluid to the extent that easy syringability exists. It should be stable under the conditions of manufacture and storage and should be preserved against the contaminating action of microorganisms, such as bacteria and fungi. The pharmaceutical carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in the appropriate solvent with various of the other ingredients enumerated above, followed by filtered sterilization. Generally, dispersions can be prepared by incorporating the various sterilized active ingredient into a sterile vehicle containing the basic dispersion medium and the required other ingredients from those enumerated above. In the case of sterile powders for the preparation of sterile injectable solutions, methods of preparation include vacuum drying and freeze drying techniques, which yield a powder of the active ingredient plus any additional desired ingredient from previously sterile filtered solution thereof.

Parenteral compositions may be formulated in dosage-unit form for ease of administration and uniformity of dosage. Dosage-unit form, as used herein, refers to physically discrete units suited as unitary dosages for the subjects to be treated, each unit containing a predetermined quantity of active material calculated to produce the desired therapeutic effect in association with the required pharmaceutical carrier. The specification for the dosage-unit forms of the present application can be chosen based upon: (a) the unique characteristics of the active material and the particular therapeutic effect to be achieved, and (b) the limitations inherent in the art of compounding such an active material for the treatment of conditions in living subjects having a condition in which bodily health is impaired as described herein.

An effective amount of a composition disclosed herein is a nontoxic, but sufficient amount of the composition, such that the desired prophylactic or therapeutic effect is produced. The exact amount of the composition that is required will vary from subject to subject, depending on the species, age, condition of the animal, severity of the inflammation or tumor-related disorder in the animal, the particular carrier or adjuvant being used, its mode of administration, and the like. Accordingly, the effective amount of any particular therapeutic composition disclosed herein will vary based on the particular circumstances, and an appropriate effective amount can be determined in each case of application by one of ordinary skill in the art using only routine experimentation.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Materials and Methods

In vivo transgene construction was carried out by homology-directed single-copy CRISPR/Cas9 gene insertion using the Mos1 mediated CRISPR Insertion (mmCRISPi) method. Briefly, transgenes were built through recombineering of 4 PCR fragments each containing at least 35 base pairs of homology. The fragments encoded a promoter, a mitochondrial targeting sequence of SDHC fused to the light activated proton pump, Mac, the red fluorescent protein mKate, and a characterized 5′ untranslated region from unc-54. The DNA coding sequence for 144 N terminal amino acids of the rat SDHC protein were fused to the N terminus of Mac by molecular cloning to achieve mitochondrial expression.

The eft-3 promoter was amplified from plasmid DNA pDD162

(forward amplification primer:
ACAGCTAGCGCACCTTTGG TCTTTTA (SEQ ID NO. 13),
reverse amplification primer:
ACAACCGGTGAGCAAAGTGTTTCCCA (SEQ ID NO. 14)).

The rab-3 promoter was amplified from plasmid DNA pSEP45

(forward amplification primer:
TCAGTGCAGTCAACATGTCGAGTTTCGTGCCGAATGACGACGACGACCTC
GACGGCAAC (SEQ ID NO. 15),
reverse amplification primer:
GCCATTTTTAAGCCTGCTTTTTTGTACAAACTTGTCTGAAAATAGGGCTA
CTGTAG (SEQ ID NO. 16)).

The vha-6 promoter was amplified from plasmid DNA pELA10

(forward amplification primer:
TCAGTGCAGTCAACATGTCGAGTTTCGTGCCGAATAGCACAGAACTGCAT
TAAG (SEQ ID NO. 17),
reverse amplification primer:
GCCATTTTTAAGCCTGCTTTTTTGTACAAACTTGTATTTTTATGGGTTTT
GGTAG (SEQ ID NO. 18)).

SDHC::Mac was amplified from plasmid DNA pBB38

(forward amplification primer:
ACAAGTTTGTACAAAAAAGCAGGCTTAAAAATGGCTGCGTTCTTGCTGAG
AC (SEQ ID NO. 19),
reverse amplification primer:
GGATCCTCCTCCTCCAGATCCTCCTCCACCTCGGGCGCCGTCGTCCTCGC
CGATC (SEQ ID NO. 20)).

mKate was amplified from plasmid DNA pAP088

(forward amplification primer:
cccgaGGTGGAGGAGGATCTGGAGGAGGAGGATCCATGGTTTCCGAGTTG
ATCAAGG (SEQ ID NO. 21),
reverse amplification primer:
TTAACGATGTCCGAGCTTGGATGGGAGATCACAATATC
(SEQ ID NO. 22)).

These PCR fragments amplified with uni 166 que homology regions, the first and fourth with homology to the genomic cut site, were microinjected into C. elegans hermaphrodite gonads with purified Cas9 protein and crRNA (GTCCGCGTTTGCTCTTTATT (SEQ ID NO. 23), DNA target) to achieve transgene construction into the genome of progeny such that gene promoters were followed by SDHC1::Mac::mKate::3′ UTR when integrated in the genome. Transgenic lines generated by mmCRISPi were outcrossed to the wild-type strain at least four times.

C. elegans Strains Growth and Maintenance

Animals were maintained at 20° C. on nematode growth medium (NGM) seeded with OP50 E. coli. All trans retinal (ATR) was added to OP50 used for seeding NGM plates for a final concentration of 100 μM where indicated, as previously described. Day 1 adult hermaphrodite animals were used for all experiments, synchronized by egg lay. Gravid adults were allowed to lay eggs for 1-2 hours and then removed from plates. The resulting synchronized populations were used for experiments at day 1 of adulthood. Transgenic strains were generated by plasmid DNA microinjection as described, and the mmCRISPi method as described above. For a complete strain list, see Table 1 below.

TABLE 1
C. elegans strains

| Strain | Genotype | Abbreviation | Source | Description |
|---|---|---|---|---|
| N2(Bristol) | | wildtype | C. elegans | Wild type |
| | | | Genetics Center | |
| | | | (CGC) | |
| APW208 | jbmSt [symbol] ::Mac::mKate:: [symbol] | mtOFF | this study | Ubiquitously expressed mitochondria-targeted light-activated |
| | [symbol] UTR * [symbol] | | | proton pump. mtOFF. |
| RB754 | [symbol] X | AMPK mutant | CGC | AAK-2 loss of function mutant from C. elegans Gene |
| | | | | KnockoutProject at the Oklahoma Medical Research Foundation. |
| APW240 | jbm [symbol] ::Mac::mKate:: [symbol] | mtOFF × AMPK | this study | which is part of the International C. elegans Gene Knockout |
| | [symbol] UTR [symbol] X | mutant | | Co [symbol] |
| | | | | APW208 crossed to RB754 to achieve mtOFF expression in an |
| | | | | AMPK loss-of-function background |
| APW254 | jbm [symbol] ::Mac::mKate:: [symbol] | mtOFF × AMPK | this study | APW240 expressing pan-neuronal AMPK |
| | [symbol] UTR [symbol] jbm [symbol] pMR729 [symbol] | mutant with | | |
| | 119p:: [symbol] ::GFP 2.5 | neuronal AMPK | | |
| | ng/ [symbol] X. | rescue | | |
| APW40 | [symbol] GFP [symbol] X | NA | (38) | Strain expressing mitochondrial intermembrane space GFP used to |
| | | | | generate APW252 |
| APW252 | jbm [symbol] ::Mac::mKate:: [symbol] | IMS::GFP × | this study | APW208 crossed with APW40 used for mtOFF localization and |
| | [symbol] UTR [symbol] | mtOFF::mKate | | topology studies |
| | [symbol] X | | | |
| APW242 | jbm [symbol] ::Mac::mKate:: [symbol] | intestinal mtOFF | this study | Strain expressing intestinal mtOFF. |
| | [symbol] UTR [symbol] | | | |
| APW258 | jbm [symbol] ::Mac::mKate:: [symbol] | neuronal mtOFF | this study | Strain expressing pan-neuronal mtOFF. |
| | [symbol] UTR [symbol] | | | |

[symbol]

indicates data missing or illegible when filed

### Fluorescence Microscopy

Images were obtained using a FV1000 Olympus laser scanning confocal microscope using a 60× oil objective (Olympus, N.A. 1.42), 561 nm diode laser illumination for red fluorescence and 488 nm for green fluorescence. Where indicated, animals were stained with 12 μM MITOTRACKER™ Green FM (Thermo Fisher Scientific, Waltham, MA) diluted in DMSO into the OP50 food for 20 hours (DMSO<0.02% final). Line scan pixel intensity was performed using ImageJ software. Fluorescent determination of mtOFF localization was performed as previously reported. Briefly, cross-section intensity plots of mitochondrial fluorescence (coexpressing either mtOFF::mKate and GFP, or mtOFF animals stained with MITOTRACKER™) were smoothed by three-point moving averages and then normalized to maximum intensity. Distance between inflection points (defined as a threshold of 10% increase in pixel intensity from the previous point, in the direction from outer border toward the middle of the mitochondrion) was measured in pixels and converted to μm.

### Mitochondria Isolation

C. elegans mitochondria were isolated from day 1 adult animals using differential centrifugation as previously described. Briefly, fed animals from 3 to 5 15-cm culture plates (˜1 million animals) were transferred into 50 mL of M9 media (22 mM KH₂PO₄, 42 mM Na₂HPO₄, 86 mM NaCl, 1 mM MgSO₄, pH 7) in a conical tube and settled by gravity on ice. Animals were rinsed with ice-cold M9 twice, then once with ice-cold mitochondrial isolation media (220 mM mannitol, 70 mM sucrose, 5 mM MOPS, 2 mM EGTA, pH 7.4) with 0.04% BSA. After again settling by gravity, the supernatant was removed, and worms were transferred onto ˜2 g of pure sea sand per 1 mL of animals in an ice-cold mortar. Animals were ground with an ice-cold pestle for 1 min and extracted from the sand using mitochondrial isolation media and transferred to a 10-mL conical tube. The samples were then homogenized in an ice-cold glass Dounce homogenizer with 40 strokes. The homogenate was centrifuged at 600 g for 5 min, then the supernatant was transferred to a new tube and centrifuged at 700 g for 10 min. The second pellet was resuspended in 1 mL of mitochondrial isolation media without BSA in a 1.5 mL tube and centrifuged at 7,000 g for 5 min. The final pellet was resuspended in 50 μL of mitochondrial isolation media without BSA. Protein was quantified using the Folin-phenol method.

Light Sources for mtOFF Activation

Illumination sources were a 580 nm Quantum SpectraLife LED Hybrid lamp by Quantum Devices, Barneveld WI (abbreviated Quantum LED in the text), a 540-600 nm GYX module, X-Cite LED1 by Excelitas, Waltham MA, (abbreviated XCite LED), and a 540-580 nm excitation filter MVX10 Fluorescence MacroZoom dissecting microscope by Olympus (abbreviated MVX) powered by an X-Cite 220 V mercury bulb by Excelitas. Light intensities are indicated for each experimental condition and were determined with a calibrated thermopile detector (818P-010-12, Newport Corporation, Irvine, CA) and optical power meter (1916-R, Newport Corporation).

### Immunoblotting

Fed adult animals were harvested in M9 media after 4 hour exposure to 1 Hz light (Quantum LED, 0.02 mW/mm²). Biological replicates were individual plates for each experimental condition, each repeated 4 times. Animals were centrifuged at 1,000 g for 1 min and ground by plastic pestle disruption in lysis buffer (20 mM Tris-HCl, 100 mM NaCl, 1 mM EDTA, 1 mM DTT, 10% glycerol, 0.1% SDS, pH 7.6, 1× Halt protease inhibitor cocktail, Thermo78429). Samples were then diluted 1:1 in sample loading buffer (100 mM Tris-HCl, 10% glycerol, 10% SDS, 0.2% w/v bromophenol blue, 2% β-mercaptoethanol). Samples were heated at 95° C. for 5 min, and 12.5 μg of protein was loaded in each lane of a 7.5% polyacrylamide gel for separation by SDS-PAGE. Protein was transferred to nitrocellulose membranes, blocked using 5% non-fat milk/TBST (50 mM Tris, 150 mM NaCl, 0.05% Tween 20, pH 8.0) for 1 h at room temperature, and incubated at 4° C. in primary antibodies diluted 1:1,000 in 5% bovine serum albumin. Membranes were washed in TBST and incubated in horseradish peroxidase-conjugated secondary antibodies for 1 h at room temperature. Antibodies used: 1:2000 anti-Actin (Abcam #ab14128), 1:10,000 anti-phospho-AMPKa Rabbit (Cell Signaling, #2535), 1:2,000 anti231 rabbit IgG (Cell Signaling #7074S), and anti-mouse IgG (Thermo Scientific #32430). Detected proteins were visualized by chemiluminescence (ChemiDoc, Bio-Rad) using ECL (Clarity Western ECL Substrate, Bio-Rad). Densitometry was performed using ImageJ software.

### Mitochondrial Membrane Potential Measurement

0.5 mg/mL isolated mitochondria were stirred in mitochondrial respiration buffer (MRB: 120 mM KCl, 25 mM sucrose, 5 mM MgCl₂, 5 mM KH₂PO₄, 1 mM EGTA, 10 mM HEPES, 1 mg/ml FF-BSA, pH 7.35) at 25° C. with 2 μM rotenone and 5 mM succinate. 20 nM tetramethylrhodamine ethyl ester (TMRE, Thermo Fisher, T669) was added to observe mitochondrial membrane potential in non-quench mode, where TMRE accumulates in the matrix and fluorescence is high in the presence of mitochondrial membrane potential. Upon addition of the protonophore FCCP, TMRE exits mitochondria and fluorescence decreases. TMRE signal was measured by Cary Eclipse Fluorescence Spectrophotometer (Agilent Technologies) using a 335-620 nm excitation filter and a 550-1,100 nm emission. Illumination was continuous throughout all measurements (0.39 mW/mm², XCite LED) with increasing light dose (fluence, J/cm²). 2 μM FCCP was added to completely depolarize mitochondria and observe minimum fluorescence. Data are normalized to maximum succinate-fueled fluorescence (F/Fmax).

### Mitochondrial Matrix pH Measurement

The BCECF-AM (Thermo Fisher, B1170), a ratiometric pH indicator, was used to measure pH change in the mitochondrial matrix in response to mtOFF activation. Isolated mitochondria (˜200 μL) were incubated with 50 μM BCECF-AM for 10 min at room temperature. Mitochondria were pelleted at 7,000 g for 5 min at 4° C., isolation media was replaced and mitochondria were pelleted again to remove residual BCECF-AM. Mitochondria were then assayed as described in the mitochondrial membrane potential measurements. 440 and 490 nm excitation wavelengths were used to measure 545 nm emission fluorescence using a Cary Eclipse Fluorescence Spectrophotometer (Agilent Technologies). Fluorescence ratio at 545 nm of 490/440 nm excitation wavelengths is presented to show pH changes in the mitochondrial matrix. Light treatment was 0.39 mW/mm² (XCite LED), and 2 μM FCCP was used at the end of each trace to establish minimum signal. Change in the ratio (ABCECF ratio) value is presented comparing before and after illumination of mtOFF.

C. elegans O₂ Consumption

Whole animal O₂ consumption was recorded using a Clark-type O₂ electrode (S1 electrode disc, DW2/2 electrode chamber and Oxy-Lab control unit, Hansatech Instruments, Norfolk UK). Adult animals were collected in M9 and pelleted by centrifugation. Animals were then rinsed in M9 and added to the chamber in 0.5 mL of continuously stirred M9. Continuous light exposure (XCite LED) was 0.39 mW/mm² throughout baseline measurement where indicated. 160 μM final concentration FCCP was added to induce maximal respiration, and 40 mM final concentration sodium azide was added to inhibit mitochondrial respiration. O₂ consumption rates (baseline, maximal, and inhibited) were measured for 10 minutes or until stable. Animals were collected after measurement for protein quantification by the Folin-phenol method.

### ATP Measurements

Whole animals were used for ATP quantification by luciferase bioluminescence. Adult animals on OP50 seeded plates were exposed to 4 hours of 1 Hz light (Quantum LED, 0.02 mW/mm²), and then collected in M9 media in 1.5 mL tubes. Samples were quickly centrifuged to pellet animals and supernatant was removed to leave pelleted animals in 100 μL M9. Samples were freeze-cracked three times in liquid nitrogen and protein concentration was measured using the Folin-phenol method. Samples were then boiled at 100° C. for 15 minutes, then placed on ice for 5 minutes. Samples were centrifuged at 14.8 g for 10 min at 4° C. An ATP determination kit was used according to the manufacturer's instructions to measure ATP levels. (Invitrogen Molecular Probes, A22066). Fold change of relative ATP levels are presented after normalization to either − or + ATR baseline levels without light.exposure.

### Whole Animal Fat Measurement

Synchronized day 1 adults were collected in M9 and pelleted by centrifugation. Animals were then rinsed in M9 and added to a glass 4 walled cuvette containing 0.5 mL M9. Animals were illuminated with 10 minutes of continuous light exposure (XCite LED, 0.39 mW/mm²) as described for O₂ consumption experiments (see C. elegans O₂ consumption section). Oil Red O (StatLab, Mckinney, TX) staining was performed as described in previous protocols. Briefly, Oil Red O product was diluted 3:2 in isopropanol, equilibrated for 2 days, and then filtered (0.4 μm). Immediately after light treatment, animals were dehydrated in 40% isopropanol/PBST (137 mM NaCl, 2.7 mM KCl, 10 mM Na₂HPO₄, 1.8 mM KH₂PO₄, Tween 20 0.1%) for 3 minutes. Animals were pelleted by centrifugation and the supernatant was removed. Animals were incubated with 600 μL of filtered Oil Red O for 2 hours at room temperature. Animals were then centrifuged, supernatant removed, and washed in PBST for 30 minutes. Animals were centrifuged and media was replaced with PBST for imaging. Animals were photographed using a MVX10 MacroZoom dissecting microscope by Olympus, and whole-animal Oil Red O density was quantified using ImageJ.

### Locomotion Measurement

The number of animal body bends in 15 seconds was scored on and off of OP50 food to assess locomotion (n=30-60 animals scored on at least 2 separate days). Change in direction of motion of the posterior pharyngeal bulb was counted as one body bend. AMPK inhibition was achieved by 24-hour exposure to 50 μM final concentration of compound C in the NGM plates. Where indicated, light treatment was continuous throughout body bend measurements (MVX, 0.265 mW/mm²).

### Hypoxia Exposure

A hypoxic chamber (Coy Laboratory Products, 5%/95% H₂/N₂ gas, palladium catalyst) was used at 26° C. with 15-50 animals per plate for hypoxia experiments. O₂ concentration was monitored and always <0.01%. 1 Hz light exposure (Quantum LED, 0.02 mW/mm²) was applied for 4 hours, 20 hours before hypoxia exposure based on a time window identified for protective signaling to occur. 18.5-hour hypoxic exposure was used to kill at least 50% of animals. 24 hours after hypoxia exposure moving animals or animals that moved in response to a light touch with an eyelash were scored as alive. Animals supplemented with ATR laid eggs onto plates without ATR that were subsequently used as adults in hypoxia experiments to minimize potential effects of ATR supplementation. Data are presented as protection (%), where baseline survival was subtracted from the survival of animals exposed to light to show potential damaging or protective effects as negative or positive values, respectively.

### Oxidative Stress Resistance

Seeded plates were supplemented with 200 mM paraquat (final concentration) 24 hours before experiments. 10-15 synchronized day 1 adult animals were moved onto paraquat plates. Control plates were kept in the dark, and experimental plates were exposed to light (Quantum LED, 1 Hz, 0.02 mW/mm²) for the duration of the experiment. Animals that were moving or those that moved in response to a light touch to the head were scored as alive. Survival was scored every hour for 7 hours.

### Statistics

Using GraphPad Prism (v7), two-tailed unpaired t-tests and one- or two-way ANOVA with post hoc tests were performed where appropriate. For hypoxia experiments, two-tailed paired t-tests were used where samples were paired by hypoxia exposure. Throughout, n values are biological replicates: independent animal populations. Technical replicates are from one biological replicate exposed separately to experimental interventions. See figure legends for detailed statistical information used.

### Example 1: Mitochondria-OFF (mtOFF) is Expressed in C. elegans Mitochondria

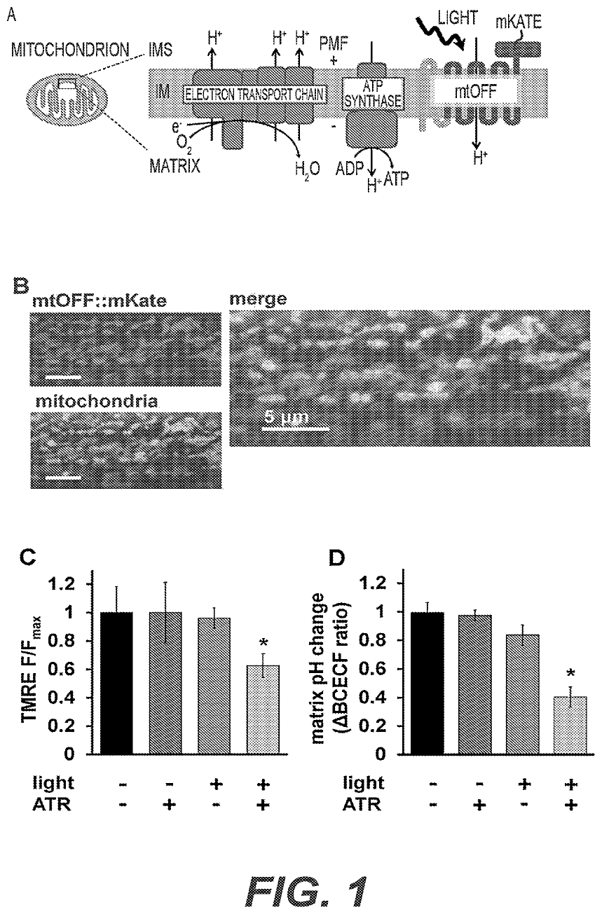

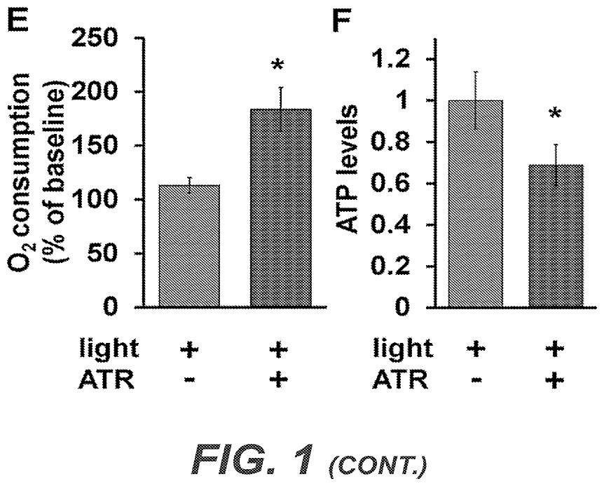

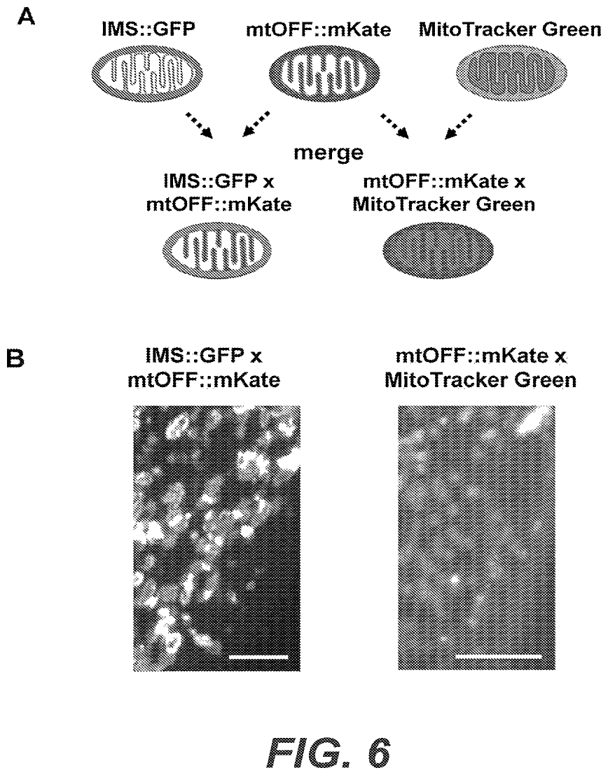

Using a ubiquitously expressed gene promoter, the light-activated proton pump, Mac, was expressed in C. elegans mitochondria. The construct was oriented to pump protons from the mitochondrial intermembrane space (IMS) into the matrix to dissipate the PMF (FIG. 1, panel A). This construct is designated mitochondria-OFF, or mtOFF, due to its ability to “turn off” mitochondrial function through the PMF in response to light, as validated here and by other studies using ChR2. The mitochondrial targeting sequence (MTS) and part of the coding sequence of the SDHC protein were used to direct and orient mitochondrial expression of Mac. This SDHC: Mac construct is the functional unit mtOFF (FIG. 1, panel A). C terminal fusion to the red fluorescent protein mKate was used for visualization in living animals. Using CRISPR/Cas9 genome editing a single copy of the mtOFF construct was integrated into the C. elegans genome under the control of a ubiquitous promoter (eft-3p) using the mmCRISPi technique to avoid over expression artifacts. Fluorescence indicated mitochondrial expression when observed by confocal microscopy in animals stained with MITOTRACKER™ Green (FIG. 1, panel B). Further, in single mitochondria of living animals, C terminal mKate fluorescence was distant from MITOTRACKER™ Green fluorescence in the mitochondrial matrix, but overlapped with IMS targeted GFP (FIG. 6, panels A-C). It was demonstrated that activation of mtOFF dissipated the PMF through both its components, Am and ΔpH (FIG. 1, panels C and D). These effects of mtOFF translated to whole animal ATP levels, whereby mtOFF activation decreased cellular energetics (FIG. 1, panels E and F). These data suggest the orientation of the proton pump was as predicted (FIG. 1, panel A) indicated by the distance between red and green fluorescence in each case (FIG. 6, panel D).

### Example 2: mtOFF Decreases Mitochondrial PMF in Response to Light

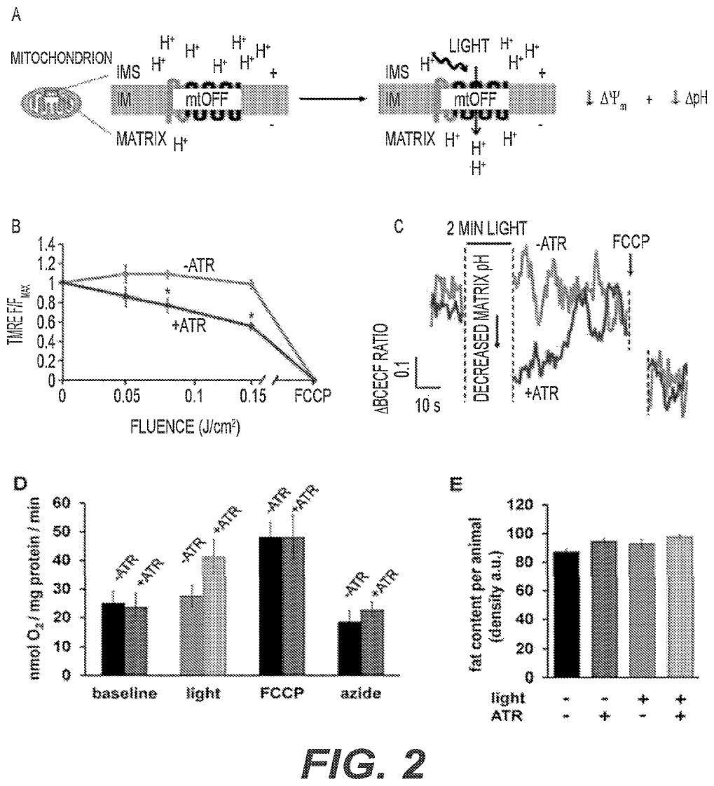

Based on the expected topology (FIG. 1, panel A) and the localization of mKate (FIG. 6, panels A-D), it was tested if mtOFF could decrease the PMF. This was tested by measuring the PMF in response to mtOFF activation in two independent assays. Optogenetic proteins such as the Mac component of mtOFF require a cofactor, all-trans retinal (ATR), for photocurrents to occur. C. elegans require ATR supplementation due to endogenous absence in the organism. Therefore, each of the experiments controlled for ATR supplementation, as well as light exposure to control for any confounding effects of light or ATR. Only light exposure and the presence of ATR allow mtOFF function. Under these conditions, mtOFF decreased the PMF in response to light when observed through both components of the gradient, the membrane potential (ΔΨm) and the pH gradient (ΔpH) (FIG. 2, panel A). Isolated mitochondria loaded with the ΔΨm fluorescent indicator tetramethylrhodamine ethyl ester (TMRE) were incubated with succinate to fuel respiration and to maintain the PMF. Upon light exposure, ΔΨm decreased significantly (FIG. 1, panel C) and light-dose dependently (FIG. 2, panel B). The ΔpH was assessed by observing BCECF fluorescence in isolated mitochondria. BCECF-AM is a ratiometric pH indicator that can be loaded into isolated mitochondria to determine changes in matrix pH. When provided succinate to maintain PMF, mtOFF activation resulted in significant, reversible matrix pH decrease (FIG. 1, panel D and FIG. 2, panel C), indicating decreased ΔpH. These data independently demonstrate that mtOFF activation dissipated the PMF through both its components, ΔΨm and ΔpH.

Respiratory control is a phenomenon of increased ETC activity in response to dissipated PMF. The ETC increases activity (and resulting 02 consumption) in attempt to maintain PMF. Therefore, it was measured whole-animal 02 consumption rates and ATP levels to assess the consequences of dissipated PMF, mtOFF activation resulted in around a 70% increase in respiration compared to control conditions (FIG. 1, panel E and FIG. 2, panel D), similar to results obtained in a cell model with mitochondria targeted ChR2. To test a whole animal measure of nutrient metabolism, we asked whether mtOFF acutely affected stored fat. mtOFF activation did not affect fat stores in whole animals (FIG. 2, panel E). The short timescale of mtOFF activation was likely not sufficient to significantly deplete stored lipids. This result suggests that the effects of mtOFF are through acute mitochondrial PMF dissipation, and that mtOFF is not affecting bulk metabolism in the short term, as expected. Relative ATP levels were then measured to assess if mtOFF was indeed interrupting respiratory control in vivo. Whole animal ATP levels were decreased after mtOFF activation (FIG. 1, panel F), indicating that mtOFF-mediated PMF dissipation decreased cellular energetics. Together, these results indicated that mtOFF caused mitochondrial uncoupling, the phenomenon of acutely increased respiration and lost ATP production because of dissipated PMF. Therefore, mtOFF functioned to decrease the PMF to alter the internal metabolic state of organisms.

### Example 3: mtOFF Modulates Energy Sensing Behavior Through Neuronal AMPK

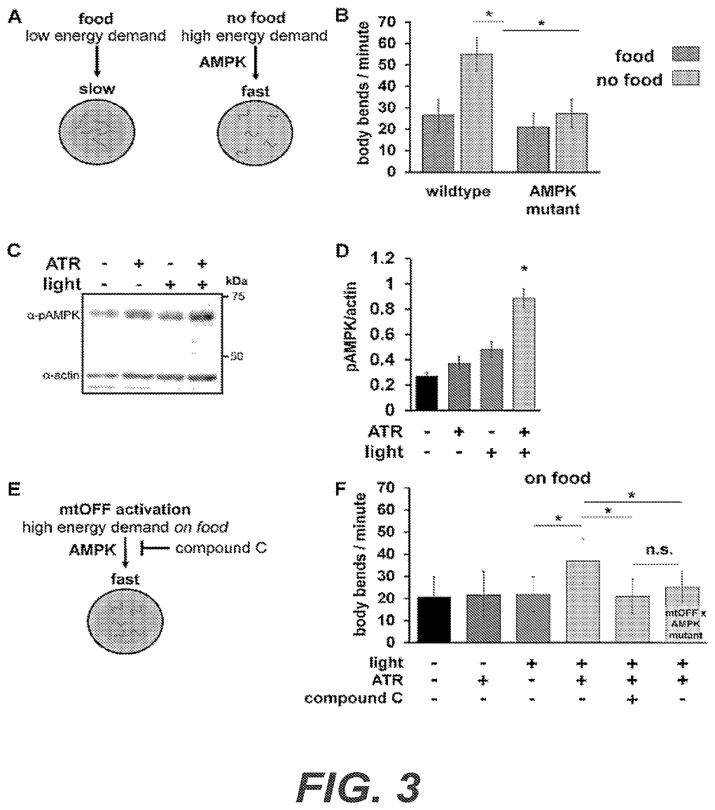

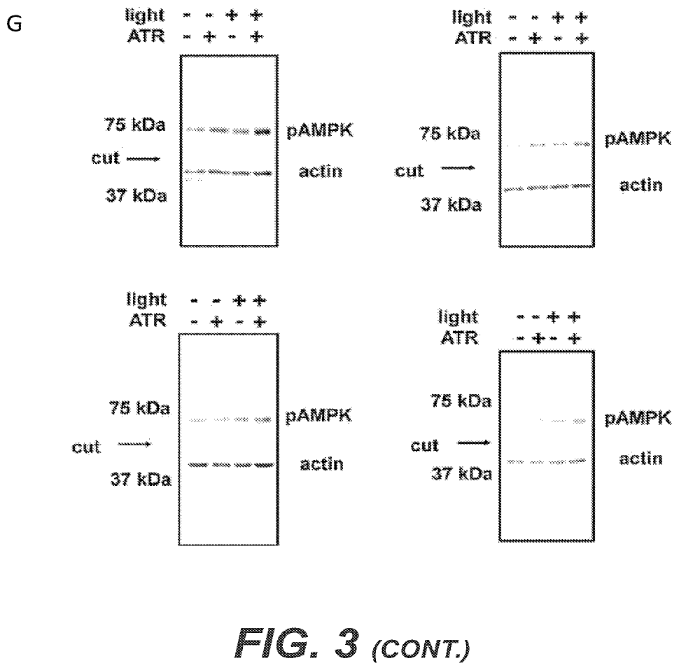

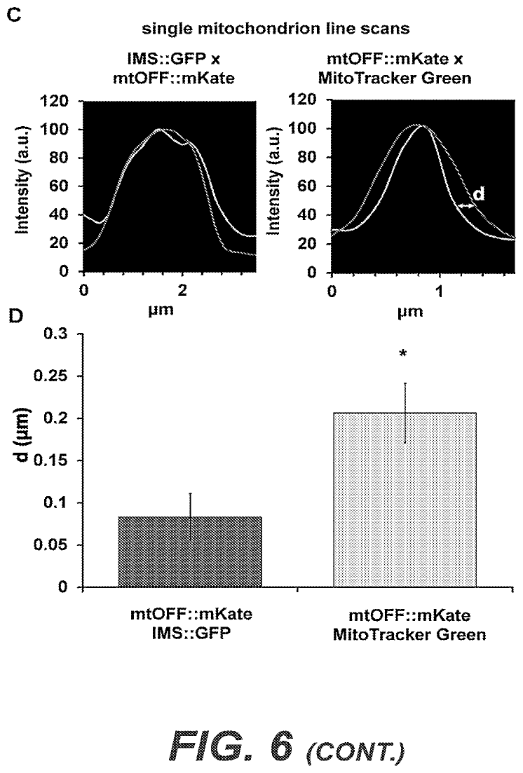

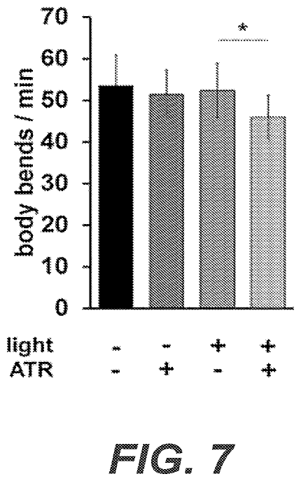

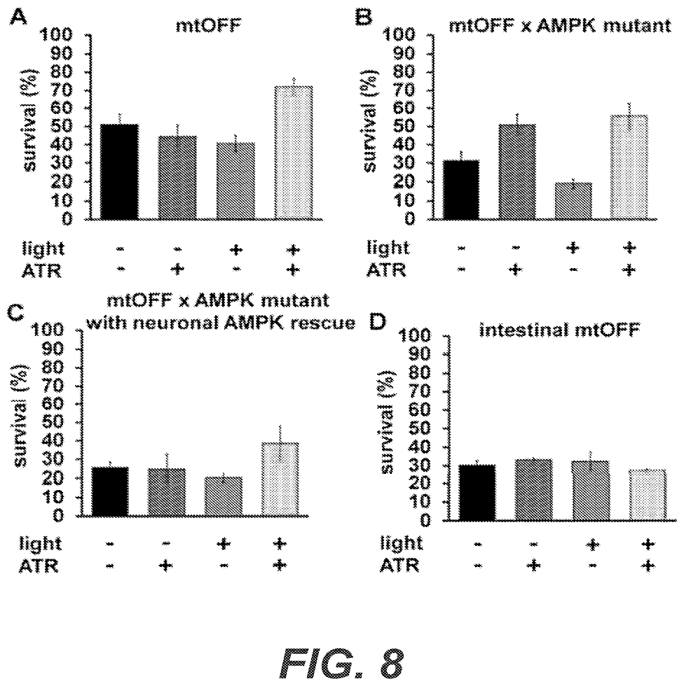

AMP-activated protein kinase (AMPK) activity is altered downstream of PMF changes and in signaling that affects C. elegans behavior. C. elegans respond to food availability and their internal metabolic state (fed versus starved) through AMPK by increasing or decreasing their locomotion speed. In the presence of food, animals will move slowly to stay in its presence, and in the absence of food, animals will increase their movement speed (FIG. 3, panels A and B). This behavior is blunted in animals lacking AMPK activity (aak-2 mutant animals ((FIG. 3, panels D). Increasing the PMF silenced AMPK signaling under starvation conditions, and could slow animal locomotion. Conversely, mtOFF activation caused increased AMPK phosphorylation and therefore activation (FIG. 3, panels C, D and G), causing increased locomotion under fed conditions (FIG. 3, panels E and F). mtOFF was able to create an energy demand resulting in increased locomotion when animals were still in the presence of food. This optogenetic effect was abolished when AMPK was inhibited with compound C. Since compound C can have non-specific or off target effects tests proceeded using genetic inhibition of AMPK. The mtOFF-mediated increase in locomotion was blocked in AMPK mutant animals expressing mtOFF (FIG. 3, panel F). These data suggest AMPK activity is regulated downstream of mitochondrial function to regulate metabolic demand, and they show that mtOFF can control animal behavior through the PMF and resultant energy sensing signaling. Since mtOFF increases locomotion speed, mtOFF was activated in the absence of food to test if there was additive effect on the high locomotion rate off of food. A small but statistically significant decrease in locomotion was found (FIG. 7) though not to the same degree as the fed rate of locomotion (FIG. 3, panel F).

AMPK signaling in neurons alone is sufficient for driving increased locomotion in response to energy demand. Therefore, it was tested whether mtOFF could trigger increased locomotion with AMPK signaling only functional in neurons to signal perceived metabolic demand.

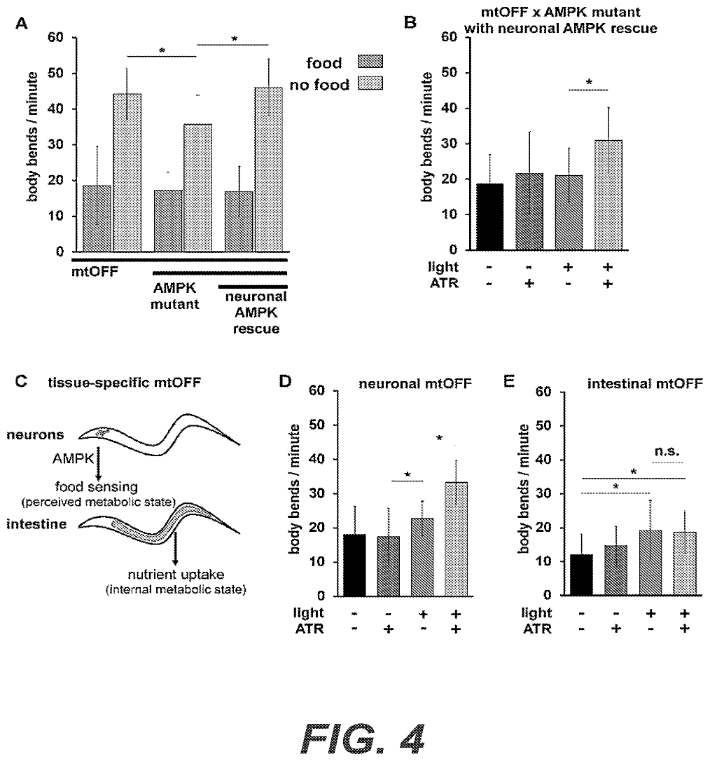

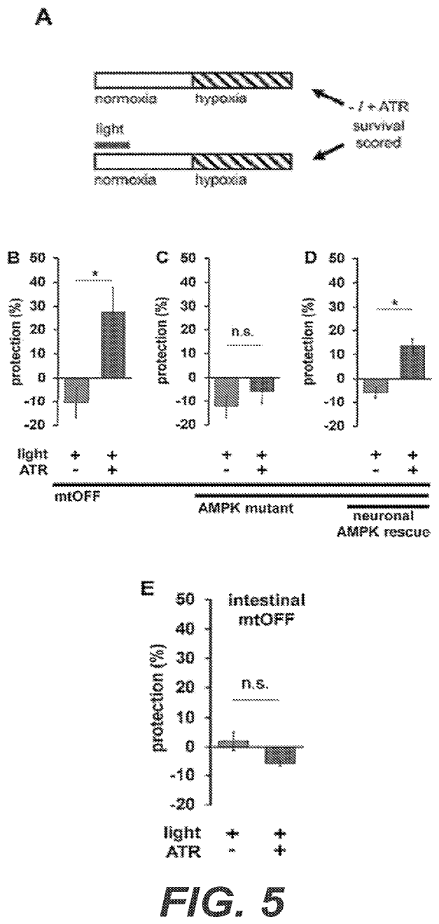

An animal's perceived or internal energetic state can influence responses to hypoxia through many mechanisms, one example being AMPK signaling in fed versus starved nutritional states (FIG. 4). Neuronal mtOFF activation protected C. elegans against impending hypoxia (FIG. 5). In animals expressing mtOFF only in intestine, mtOFF activation did not confer protection against hypoxia (FIG. 5, panels E). These data show acute PMF loss in intestine specifically is not sufficient for hypoxia resistance. This supports a model in which perceived metabolic state (through neuronal AMPK) can act to trigger organism-wide protection.

Using a pan-neuronal gene promoter, neuronal AMPK expression was rescued in AMPK mutant animals as previously described and confirmed its sufficiency for restoring increased locomotion upon starvation. The AMPK mutant strain alone and the mtOFF AMPK mutant strain in the absence of food appeared to have baseline differences, however, tests did not compare these effects directly. Regardless, AMPK mutant animals expressing mtOFF did not respond to starvation by increasing locomotion to the same degree as wildtype, unless AMPK was rescued in neurons (FIG. 4, panel A). Under fed conditions, mtOFF activation increased locomotion in AMPK mutant animals with rescued neuronal AMPK (FIG. 4, panel B), similar to the results with mtOFF in a wildtype background. This confirmed that neuronal AMPK alone was sufficient to respond to dissipated PMF. Following this result in neurons, it was asked whether tissue-restricted mtOFF activity could have similar effects on locomotion. This hypothesis was tested using tissue-specific gene promoters to drive mtOFF expression separately in neurons and intestine (FIG. 4, panel C). Intestinal expression of mtOFF was tested to rule out the possibility of signaling coming from intestine, the organ that may sense energy availability through its nutrient-absorbing function, creating the internal metabolic state (fed versus starved). Using a pan-neuronal gene promoter (rab-3p) and an intestinal gene promoter (vha-6p) to drive mtOFF expression, it was found that neuronal mtOFF activation was sufficient to trigger increased locomotion (FIG. 4, panel D), but intestinal mtOFF activation was not (FIG. 4, panel E). In these experiments, light exposure alone resulted in small increases in locomotion, however, under conditions of active mtOFF (light plus ATR) neuronal mtOFF increased locomotion compared to all controls. These results indicate that mitochondrial function and energy sensing through AMPK are tightly linked and especially important in neurons. This tight spatial control of mitochondria and subsequent control of behavior shows that communication throughout the cell can be traced back to PMF changes in mitochondria.

### Example 4: mtOFF Protects Against Hypoxia Through Neuronal AMPK

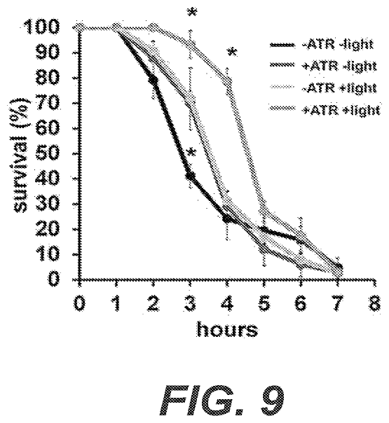

Because the optogenetic approach allows for spatiotemporal control of mitochondrial function, the ability of prophylactic PMF dissipation to protect against hypoxia was tested (FIG. 5, panels A) and it was asked if neuronal AMPK could be specifically involved. Chronic PMF dissipation through pharmacology and genetics protects against hypoxia, however, PMF dissipation may only be required before hypoxia exposure for some mechanisms of resistance. Therefore, it was tested if mtOFF activation could protect C. elegans against impending hypoxia. Consistent with mammalian cell models, mtOFF activation to dissipate the PMF was also protective in the C. elegans model (FIG. 5, panels B) when the percent of increased survival after hypoxia and light treatment was measured, either with or without ATR. PMF dissipation during oxidative stress is protective in many models. Therefore, to expand the physiologic implications of mtOFF activity, it was tested if chronic mtOFF activation during oxidative stress could improve survival. Paraquat causes oxidative damage and death in C. elegans, mimicking oxidative stress that occurs upon reoxygenation after hypoxia. When exposed to a toxic dose of paraquat, mtOFF activation resulted in increased survival over time, as expected (FIG. 9). This result shows how mtOFF activity impacts a general physiologic paradigm of oxidant mediated damage at the whole animal level.

In addition, AMPK was required for prophylactic hypoxia protection, as mtOFF activation in AMPK mutant animals was not protective (FIG. 5, panel C). It was then tested if neuron-specific AMPK rescue by extrachromosomal array expression could restore protection, and found that mtOFF activation was again protective (FIG. 5, panel D). The apparent partial rescue may be attributed to the heterologous promoter expression (rab-3p) of AMPK. These data indicated neuronal AMPK activity was sufficient for hypoxia resistance. This supports a model in which perceived metabolic state (through neuronal AMPK) can act to trigger organism wide protection.

In lifespan studies, and in the context of mitochondrial proteostatic stress, intestinal mitochondria mediate robust stress resistance which can act in the absence of neuronal mitochondrial dysfunction. Therefore it was tested if direct intestinal PMF dissipation could result in resistance against hypoxia. In animals expressing mtOFF only in intestine, mtOFF activation did not confer protection against hypoxia (FIG. 5, panel E). These data show acute PMF loss in intestine specifically is not sufficient for hypoxia resistance. While proteostatic maintenance in mitochondria mediates stress resistance within intestine, direct PMF changes do not play a role in this tissue. This result suggests that in the absence of perceived energy crisis through neuronal AMPK activity, there is also absence of organism-wide stress resistance. In summary, the data suggest that PMF dissipation is a trigger for AMPK-mediated hypoxia resistance, and that neuronal AMPK is a driver of whole organism protection.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed:

1. A fusion protein comprising:
a first moiety that targets the fusion protein to the mitochondrial inner membrane, and
a second moiety that comprises a light-activated proton pump,
wherein the first moiety orients the light-activated proton pump in the direction to pump protons from the inner membrane space to the mitochondrial matrix.

2. The fusion protein of claim 1, wherein the first moiety comprises an amino acid sequence selected from the group consisting of the mitochondria targeting sequence and transmembrane domains of one of human succinate dehydrogenase cytochrome b560 subunit (SDHC), mouse SDHC and rat SDHC.

3. The fusion protein of claim 1, wherein the first moiety comprises SEQ ID NO:8.

4. The fusion protein of claim 1, wherein the second moiety comprises an amino acid sequence selected from the group consisting of the protein sequence of Mac and variants, Arch and variants, bacteriorhodopsin (bR) and delta rhodopsin (dR).

5. The fusion protein of claim 1, wherein the second moiety comprises SEQ ID NO: 10.

6. The fusion protein of claim 1, wherein the first moiety is linked to the second moiety through a peptide linker.

7. The fusion protein of claim 6, wherein peptide linker comprises a sequence of pro-ala-gly.

8. The fusion protein of claim 1, further comprising a third moiety that functions as a detection marker.

9. The fusion protein of claim 1, wherein the first moiety comprises an amino acid sequence that is at least 80% homologous to SEQ ID NO:8 and wherein the second moiety comprises an amino acid sequence that is at least 80% homologous to SEQ ID NO:10.

10. The fusion protein of claim 9, comprising the amino acid sequence of SEQ ID NO: 11.

11. A polynucleotide encoding the fusion protein of claim 1.

12. The polynucleotide of claim 11, comprising the nucleotide sequence of SEQ ID NO: 12.

13. An expression cassette comprising:
the polynucleotide of claim 11; and
a regulatory sequence operably linked to the polynucleotide.

14. An expression vector comprising the polynucleotide of claim 11.

15. A mitochondria comprising the fusion protein of claim 1.

16. A cell comprising the mitochondria of claim 15.

17. A pharmaceutical composition, comprising:
the expression vector of claim 14; and
a pharmaceutically acceptable carrier.

18. A method of treating or ameliorating symptoms of neurodegenerative diseases in a subject, comprising the steps of:
expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix;
exposing the target cells to light to activate the proton pump to decrease the protonmotive force (PMF) across the mitochondrial inner membrane,
wherein decreased PMF in the mitochondria of the target cells prevents development of symptoms, or ameliorates existing symptoms, of neurodegenerative diseases.

19. The method of claim 18, wherein the target cells are neuronal cells.

20. The method of claim 18, wherein the fusion protein is expressed in the target cells by infecting the target cells with a viral vector capable of expressing the fusion protein in the target cells.

21. A method of enhancing cell resistance to hypoxia in a subject, comprising the steps of:
expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix;
exposing the target cells to light to activate the proton pump to decrease the protonmotive force (PMF) across the mitochondrial inner membrane,
wherein decreased PMF in the mitochondria of the target cells enhances the target cells' resistance to hypoxia.

22. The method of claim 21, wherein the target cells are neuronal cells.

23. The method of claim 20, wherein the fusion protein is expressed in the target cells by infecting the target cells with a viral vector capable of expressing the fusion protein in the target cells.

24. A method of enhancing cell resistance to stress in a subject, comprising the steps of:
expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix, wherein the first moiety comprises a targeting/orienting sequence from SDHC;
exposing the target cells to light to activate the proton pump to decrease the protonmotive force (PMF) across the mitochondrial inner membrane,
wherein decreased PMF in mitochondria of the target cells enhances the target cells' resistance to stress.

25. The method of claim 24, wherein PMF in mitochondria of the target cells is decreased to an extent that results in mitochondria autophagy.

26. The method of claim 24, wherein the light-activated proton pump in the second moiety is a Mac proton pump.

27. A method of treating or ameliorating symptoms of metabolic disorders or conditions caused by mitochondrial dysfunction in a subject, comprising the steps of:
expressing a fusion protein in target cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix, wherein the first moiety comprises a targeting/orienting sequence from SDHC;
exposing the target cells to light to activate the proton pump to decrease the protonmotive force (PMF) across the mitochondrial inner membrane,
wherein decreased PMF in mitochondria of the target cells prevents development symptoms, or ameliorating existing symptoms, of the metabolic disorder.

28. The method of claim 27, wherein PMF in mitochondria of the target cells is decreased to an extent that results in mitochondria autophagy.

29. The method of claim 27, wherein the light-activated proton pump in the second moiety is a Mac proton pump.

30. The method of claim 27, wherein the metabolic disorder or conditions caused by mitochondrial dysfunction in a subject is one or more selected from a group comprising psoriasis, skin inflammation, muscle hypertonicity, and fungal infection.

31. A method of treating cancer in a subject suffering from cancer, comprising the steps of:
expressing a fusion protein in target cancer cells in the subject, wherein the fusion protein comprises a first moiety that targets the fusion protein to the mitochondrial inner membrane, and a second moiety that comprises a light-activated proton pump, wherein the first moiety orients the light-activated proton pump in the direction to pump protons from the inner membrane space to the mitochondrial matrix;
exposing target cancer cells to light to activate the proton pump to decrease the protonmotive force (PMF) across the mitochondrial inner membrane,
wherein decreased PMF in mitochondria of the target cancer cells inhibits cancer cell growth in the subject.

32. The method of claim 31, wherein PMF in mitochondria of the target cancer cells is decreased to an extent that results in mitochondria autophagy.


SEQUENCE LISTING (23 sequences)

SEQ ID NO: 1 - protein, 169 aa, Homo sapiens
  misc_feature (1)..(169): Human SDHC (UniProtKB - Q99643)
MAALLLRHVGRHCLRAHFSPQLCIRNAVPLGTTAKEEMERFWNKNIGSNRPLSPHITIYSWSLPMAMSICHRGTGIALSAGVSLFGMSALLLPGNFESYLELVKSLCLGPALIHTAKFALVFPLMYHTWNGIRHLMWDLGKGLKIPQLYQSGVVVLVLTVLSSMGLAAM

SEQ ID NO: 2 - protein, 138 aa, Homo sapiens
  misc_feature (1)..(138): first 138 aa of human SDHC sequence
MAALLLRHVGRHCLRAHFSPQLCIRNAVPLGTTAKEEMERFWNKNIGSNRPLSPHITIYSWSLPMAMSICHRGTGIALSAGVSLFGMSALLLPGNFESYLELVKSLCLGPALIHTAKFALVFPLMYHTWNGIRHLMWD

SEQ ID NO: 3 - protein, 144 aa, Homo sapiens
  misc_feature (1)..(144): first 144 aa of human SDHC sequence
MAALLLRHVGRHCLRAHFSPQLCIRNAVPLGTTAKEEMERFWNKNIGSNRPLSPHITIYSWSLPMAMSICHRGTGIALSAGVSLFGMSALLLPGNFESYLELVKSLCLGPALIHTAKFALVFPLMYHTWNGIRHLMWDLGKGLK

SEQ ID NO: 4 - protein, 169 aa, Mus musculus
  misc_feature (1)..(169): Mouse SDHC
MAAFLLRHVSRHCLRAHLNAQLCIRNAAPLGTTAKEEMERFWKKNTSSNRPLSPHLTIYKWSLPMALSVCHRGSGIALSGGVSLFGLSALLLPGNFESYLMFVKSLCLGPTLIYSAKFVLVFPLMYHSLNGIRHLLWDLGKGLAIPQVWLSGVAVVVLAVLSSGGLAAL

SEQ ID NO: 5 - protein, 138 aa, Mus musculus
  misc_feature (1)..(138): first 138 aa of mouse SDHC sequence
MAAFLLRHVSRHCLRAHLNAQLCIRNAAPLGTTAKEEMERFWKKNTSSNRPLSPHLTIYKWSLPMALSVCHRGSGIALSGGVSLFGLSALLLPGNFESYLMFVKSLCLGPTLIYSAKFVLVFPLMYHSLNGIRHLLWD

SEQ ID NO: 6 - protein, 144 aa, Mus musculus
  misc_feature (1)..(144): first 144 aa of mouse SDHC sequence
MAAFLLRHVSRHCLRAHLNAQLCIRNAAPLGTTAKEEMERFWKKNTSSNRPLSPHLTIYKWSLPMALSVCHRGSGIALSGGVSLFGLSALLLPGNFESYLMFVKSLCLGPTLIYSAKFVLVFPLMYHSLNGIRHLLWDLGKGLA

SEQ ID NO: 7 - protein, 169 aa, Rattus norvegicus
  misc_feature (1)..(169): Rat SDHC (UniProtKB -Q641Z9)
MAALLLRHIGRHCLRAHLSSQLCIRNAAPLGTTAKEEMARFWNKNTSSNRPVSPHLTIYRWSLPMAMSVCHRGSGIAMSGGVSLFGLSALLLPGNFESYLMLVKSLCLGPALIHAAKFVLVFPLMYHSLNGVRHLMWDLGKGLSISQVQLSGVTVLVLAVLSSAGLAAI

SEQ ID NO: 8 - protein, 138 aa, Rattus norvegicus
  misc_feature (1)..(138): first 138 aa of rat SDHC sequence
MAAFLLRHIGRHCLRAHLSSQLCIRNAAPLGTTAKEEMARFWNKNTSSNRPVSPHLTIYRWSLPMAMSVCHRGSGIAMSGGVSLFGLSALLLPGNFESYLMLVKSLCLGPALIHAAKFVLVFPLMYHSLNGVRHLMWD

SEQ ID NO: 9 - protein, 144 aa, Rattus norvegicus
  misc_feature (1)..(144): first 144 aa of rat SDHC sequence
MAAFLLRHIGRHCLRAHLSSQLCIRNAAPLGTTAKEEMARFWNKNTSSNRPVSPHLTIYRWSLPMAMSVCHRGSGIAMSGGVSLFGLSALLLPGNFESYLMLVKSLCLGPALIHAAKFVLVFPLMYHSLNGVRHLMWDLGKGLA

SEQ ID NO: 10 - protein, 314 aa, L. maculans
  misc_feature (1)..(314): Mac (L. maculans) sequence
LIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGAR

SEQ ID NO: 11 - protein, 461 aa, Artificial Sequence
  Synthetic Complete mtOFF fusion protein sequence
MAAFLLRHIGRHCLRAHLSSQLCIRNAAPLGTTAKEEMARFWNKNTSSNRPVSPHLTIYRWSLPMAMSVCHRGSGIAMSGGVSLFGLSALLLPGNFESYLMLVKSLCLGPALIHAAKFVLVFPLMYHSLNGVRHLMWDLGKGLAPAGLIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGAR

SEQ ID NO: 12 - DNA, 1383 bp, Artificial Sequence
  Synthetic mtOFF nucleotide sequence
atggctgcgttcttgctgagacacatcggccgccattgcctccgagcccaccttagttctcagctctgtatcagaaatgctgctcctttgggaaccacagctaaggaagaaatggcacggttctggaataagaacacgagttccaaccgtcctgtctctccccatttgactatctacaggtggtctcttcccatggcaatgtctgtttgccaccgaggctctgggatagccatgagtggaggggtctctctttttggcctgtcggcactgctgcttcctgggaactttgagtcgtatctgatgcttgtgaagtccctgtgtttggggccagcgctgatccatgcagccaagttcgtgcttgtctttcctctcatgtaccactcattgaatggggtccgacacttgatgtgggacctaggaaaaggcctggcacctgcaggcttgatcgtggaccagttcgaggaggtgctgatgaagaccagccagctgttcccactgccaaccgctacccagagcgcccagccaacccacgtggcccccgtgccaaccgtgctgcccgacacccccatctacgagaccgtgggcgacagcggcagcaagaccctgtgggtggtgttcgtgctgatgctgatcgccagcgccgccttcaccgccctgagctggaagatccccgtgaacaggaggctgtaccacgtgatcaccaccatcatcaccctgaccgccgccctgagctacttcgctatggctaccggccacggagtggccctgaacaagatcgtgatcaggacccagcacgaccacgtgcccgacacctacgagaccgtgtaccgacaggtgtactacgccaggtacatcgactgggctatcaccaccccactgctgctgctggacctgggactgctggctggaatgagcggagcccacatcttcatggccatcgtggctgacctgatcatggtgctgaccggcctgttcgctgctttcggcagcgagggaaccccacagaagtggggatggtacaccatcgcctgcatcgcctacatcttcgtggtgtggcacctggtgctgaacggcggcgccaacgccagggtgaagggcgagaagctgaggagcttcttcgtggccatcggagcttacaccctgatcctgtggaccgcttacccaatcgtgtggggactggctgacggagctaggaagatcggagtggacggagagatcatcgcttacgctgtgctggacgtgctggctaagggagtgttcggagcttggctgctggtgacccacgccaacctgagggagagcgacgtggagctgaacggcttctgggccaacggcctgaacagggagggcgccatcaggatcggcgaggacgacggcgcccga

SEQ ID NO: 13 - DNA, 26 bp, Artificial Sequence
  Synthetic Forward amplification primer for promoter eft-3
acagctagcgcacctttggtctttta

SEQ ID NO: 14 - DNA, 26 bp, Artificial Sequence
  Synthetic Reverse amplification primer for promoter eft-3
acaaccggtgagcaaagtgtttccca

SEQ ID NO: 15 - DNA, 59 bp, Artificial Sequence
  Synthetic Forward amplification primer for promoter rab-3
tcagtgcagtcaacatgtcgagtttcgtgccgaatgacgacgacgacctcgacggcaac

SEQ ID NO: 16 - DNA, 56 bp, Artificial Sequence
  Synthetic Reverse amplification primer for promoter rab-3
gccatttttaagcctgcttttttgtacaaacttgtctgaaaatagggctactgtag

SEQ ID NO: 17 - DNA, 54 bp, Artificial Sequence
  Synthetic Forward amplification primer for promoter vha-6
tcagtgcagtcaacatgtcgagtttcgtgccgaatagcacagaactgcattaag

SEQ ID NO: 18 - DNA, 55 bp, Artificial Sequence
  Synthetic Reverse amplification primer for promoter vha-6
gccatttttaagcctgcttttttgtacaaacttgtatttttatgggttttggtag

SEQ ID NO: 19 - DNA, 52 bp, Artificial Sequence
  Synthetic Forward amplification primer for SDHC1::Mac
acaagtttgtacaaaaaagcaggcttaaaaatggctgcgttcttgctgagac

SEQ ID NO: 20 - DNA, 55 bp, Artificial Sequence
  Synthetic Reverse amplification primer for SDHC1::Mac
ggatcctcctcctccagatcctcctccacctcgggcgccgtcgtcctcgccgatc

SEQ ID NO: 21 - DNA, 57 bp, Artificial Sequence
  Synthetic Forward amplification primer for red fluorescent protein mKate
cccgaggtggaggaggatctggaggaggaggatccatggtttccgagttgatcaagg

SEQ ID NO: 22 - DNA, 38 bp, Artificial Sequence
  Synthetic Reverse amplification primer for red fluorescent protein mKate
ttaacgatgtccgagcttggatgggagatcacaatatc

SEQ ID NO: 23 - DNA, 20 bp, Artificial Sequence
  Synthetic DNA target of crRNA
gtccgcgtttgctctttatt